SEQUENCE LISTING (61 sequences)

SEQ ID NO: 1 - protein, 122 aa, Mus musculus
QVKLQQSGPELVRPGTSVKVSCTASGDAFTNYLIEWVKQRPGQGLEWIGLIYPDSGYINYNENFKGKATLTADRSSSTAYMQLSSLTSEDSAVYFCARRFAYYGSGYYFDYWGQGTTLTVSS

SEQ ID NO: 2 - protein, 112 aa, Mus musculus
DVVMTQTPLSLPVSLGDQASISCRSSQSLLKTNGNTYLHWYLQKPGQSPKLLIFKVSNRFSGVPDRFSGSGSGTDFTLKISRVEAEDLGVYFCSQSTHFPFTFGTGTKLEIK

SEQ ID NO: 3 - protein, 122 aa, Mus musculus
QVQLQQSGAELVRPGTSVKVSCKASGYGFINYLIEWIKQRPGQGLEWIGLINPGSDYTNYNENFKGKATLTADKSSSTAYMHLSSLTSEDSAVYFCARRFGYYGSGNYFDYWGQGTTLTVSS

SEQ ID NO: 4 - protein, 112 aa, Mus musculus
DVVMTQTPLSLPVSLGDQASISCTSGQSLVHINGNTYLHWYLQKPGQSPKLLIYKVSNLFSGVPDRFSGSGSGTDFTLKISRVEAEDLGVYFCSQSTHFPFTFGTGTKLEIK

SEQ ID NO: 5 - protein, 122 aa, Mus musculus
QVQLQQSGAELVRPGTSVKVSCKASGDAFTNYLIEWVKQRPGQGLEWIGLIIPGTGYTNYNENFKGKATLTADKSSSTAYMQLSSLTSEDSAVYFCARRFGYYGSSNYFDYWGQGTTLTVSS

SEQ ID NO: 6 - protein, 112 aa, Mus musculus
DVVMTQTPLSLPVSLGDQASISCRSSQSLVHSNGNTYLHWYLQKPGQSPKLLIYKVSNRFSGVPDRFSGSGPGTDFTLKISRVEAEDLGIYFCSQSTHFPFTFGTGTKLEIK

SEQ ID NO: 7 - protein, 122 aa, Mus musculus
QVQLQQSGAELVRPGTSVKLSCKASGDAFTNYLIEWVKQRPGQGLEWIGLIIPGTGYTNYNENFKGKATLTADKSSSTAYMQLSSLTSEDSAVYFCARRFGYYGSGYYFDYWGQGTTLTVSS

SEQ ID NO: 8 - protein, 112 aa, Mus musculus
DVVMTQTPLSLPVSLGDQASISCRSSQSLVHSNGNTYLHWYLQKPGQSPKLLIYKVSNRFSGVPDRFSGSGPGTDFTLKISRVEAEDLGVYFCSQSTHFPFTFGTGTKLEIK

SEQ ID NO: 9 - protein, 118 aa, Mus musculus
DIQLQESGPGLVKPSQSLSLTCSVTGFSITSGYYWTWIRQFPGNKLEWVAYIGYDGSNDSNPSLKNRISITRDTSKNQFFLKLNSVTTEDTATYYCARAMLRRGFDYWGQGTTLTVSS

SEQ ID NO: 10 - protein, 104 aa, Mus musculus
QIVLTQSPAIMSASPGEKVTMTCSASSSLSYMHWYQQKPGTSPKRWIYDTSKLASGVPARFSGSGSGTSYSLTISSMEAEDAATYYCHRRSSYTFGGGTKLEIK

SEQ ID NO: 11 - protein, 10 aa, Mus musculus
GDAFTNYLIE

SEQ ID NO: 12 - protein, 17 aa, Mus musculus
LIYPDSGYINYNENFKG

SEQ ID NO: 13 - protein, 13 aa, Mus musculus
RFAYYGSGYYFDY

SEQ ID NO: 14 - protein, 16 aa, Mus musculus
RSSQSLLKTNGNTYLH

SEQ ID NO: 15 - protein, 7 aa, Mus musculus
KVSNRFS

SEQ ID NO: 16 - protein, 9 aa, Mus musculus
SQSTHFPFT

SEQ ID NO: 17 - protein, 5 aa, Mus musculus
NYLIE

SEQ ID NO: 18 - protein, 10 aa, Mus musculus
GYGFINYLIE

SEQ ID NO: 19 - protein, 17 aa, Mus musculus
LINPGSDYTNYNENFKG

SEQ ID NO: 20 - protein, 13 aa, Mus musculus
RFGYYGSGNYFDY

SEQ ID NO: 21 - protein, 16 aa, Mus musculus
TSGQSLVHINGNTYLH

SEQ ID NO: 22 - protein, 7 aa, Mus musculus
KVSNLFS

SEQ ID NO: 23 - protein, 17 aa, Mus musculus
LIIPGTGYTNYNENFKG

SEQ ID NO: 24 - protein, 13 aa, Mus musculus
RFGYYGSSNYFDY

SEQ ID NO: 25 - protein, 16 aa, Mus musculus
RSSQSLVHSNGNTYLH

SEQ ID NO: 26 - protein, 13 aa, Mus musculus
RFGYYGSGYYFDY

SEQ ID NO: 27 - protein, 11 aa, Mus musculus
GFSITSGYYWT

SEQ ID NO: 28 - protein, 16 aa, Mus musculus
YIGYDGSNDSNPSLKN

SEQ ID NO: 29 - protein, 9 aa, Mus musculus
AMLRRGFDY

SEQ ID NO: 30 - protein, 10 aa, Mus musculus
SASSSLSYMH

SEQ ID NO: 31 - protein, 7 aa, Mus musculus
DTSKLAS

SEQ ID NO: 32 - protein, 7 aa, Mus musculus
HRRSSYT

SEQ ID NO: 33 - protein, 6 aa, Mus musculus
SGYYWT

SEQ ID NO: 34 - protein, 112 aa, Artificial sequence
  Humanized antibody sequence
DIVMTQTPLSLPVTPGEPASISCTSGQSLVHINGNTYLHWYLQKPGQSPQLLIYKVSNLFSGVPDRFSGSGSGTDFTLKISRVEAEDVGVYYCSQSTHFPFTFGQGTKLEIK

SEQ ID NO: 35 - protein, 112 aa, Artificial sequence
  Humanized antibody sequence
DVVMTQTPLSLPVTPGEPASISCTSGQSLVHINGNTYLHWYLQKPGQSPKLLIYKVSNLFSGVPDRFSGSGSGTDFTLKISRVEAEDVGVYFCSQSTHFPFTFGQGTKLEIK

SEQ ID NO: 36 - protein, 112 aa, Artificial sequence
  Humanized antibody sequence
DIVMTQTPLSLPVTPGEPASISCTSGQSLVHINGNTYLHWYLQKPGQSPKLLIYKVSNLFSGVPDRFSGSGSGTDFTLKISRVEAEDVGVYFCSQSTHFPFTFGQGTKLEIK

SEQ ID NO: 37 - protein, 112 aa, Artificial sequence
  Humanized antibody sequence
DVVMTQTPLSLPVTPGEPASISCTSGQSLVHINGNTYLHWYLQKPGQSPQLLIYKVSNLFSGVPDRFSGSGSGTDFTLKISRVEAEDVGVYFCSQSTHFPFTFGQGTKLEIK

SEQ ID NO: 38 - protein, 112 aa, Artificial sequence
  Humanized antibody sequence
DVVMTQTPLSLPVTPGEPASISCTSGQSLVHINGNTYLHWYLQKPGQSPKLLIYKVSNLFSGVPDRFSGSGSGTDFTLKISRVEAEDVGVYYCSQSTHFPFTFGQGTKLEIK

SEQ ID NO: 39 - protein, 112 aa, Artificial sequence
  Humanized antibody sequence
DVVMTQTPLSLPVTPGEPASISCTSGQSLVHINGNTYLHWYLQKPGQSPQLLIYKVSNLFSGVPDRFSGSGSGTDFTLKISRVEAEDVGVYYCSQSTHFPFTFGQGTKLEIK

SEQ ID NO: 40 - protein, 112 aa, Artificial sequence
  Humanized antibody sequence
DVVMTQTPLSLPVTPGEPASISCRSSQSLLKTNGNTYLHWYLQKPGQSPKLLIYKVSNRFSGVPDRFSGSGSGTDFTLKISRVEAEDVGVYFCSQSTHFPFTFGQGTKLEIK

SEQ ID NO: 41 - protein, 112 aa, Artificial sequence
  Humanized antibody sequence
DVVMTQTPLSLPVTPGEPASISCRSSQSLLKTNGNTYLHWYLQKPGQSPQLLIYKVSNRFSGVPDRFSGSGSGTDFTLKISRVEAEDVGVYYCSQSTHFPFTFGQGTKLEIK

SEQ ID NO: 42 - protein, 112 aa, Artificial sequence
  Humanized antibody sequence
DVVMTQTPLSLPVTPGEPASISCRSSQSLLKTNGNTYLHWYLQKPGQSPKLLIFKVSNRFSGVPDRFSGSGSGTDFTLKISRVEAEDVGVYFCSQSTHFPFTFGQGTKLEIK

SEQ ID NO: 43 - protein, 122 aa, Artificial sequence
  Humanized antibody sequence
EVQLVQSGAEVKKPGESLKISCQSFGYIFINYLIEWVRQMPGQGLEWMGLINPGSDYTNYNENFKGQVTISADKSSSTAYLQWSSLKASDTAMYFCARRFGYYGSGNYFDYWGQGTMVTVSS

SEQ ID NO: 44 - protein, 122 aa, Artificial sequence
  Humanized antibody sequence
EVQLVQSGAEVKKPGESLKISCQAFGYGFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 45 - protein, 122 aa, Artificial sequence
  Humanized antibody sequence
EVQLVQSGAEVKKPGESLKISCQSFGYGFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 46 - protein, 122 aa, Artificial sequence
  Humanized antibody sequence
EVQLVQSGAEVKKPGESLKISCQAFGYIFINYLIEWIRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 47 - protein, 122 aa, Artificial sequence
  Humanized antibody sequence
EVQLVQSGAEVKKPGESLKISCQAFGYGFINYLIEWVRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 48 - protein, 122 aa, Artificial sequence
  Humanized antibody sequence
EVQLVQSGAEVKKPGESLKISCQAFGYGFINYLIEWIRQMPGQGLEWMGLINPGSDYTNYNENFKGQATLSADKSSSTAYLQWSSLKASDTAMYFCARRFGYYGSGNYFDYWGQGTMVTVSS

SEQ ID NO: 49 - protein, 122 aa, Artificial sequence
  Humanized antibody sequence
EVQLVQSGAEVKKPGESLKISCQAFGYGFINYLIEWIRQMPGQGLEWIGLINPGSDYTNYNENFKGQVTLSADKSSSTAYLQWSSLKASDTAMYFCARRFGYYGSGNYFDYWGQGTMVTVSS

SEQ ID NO: 50 - protein, 122 aa, Artificial sequence
  Humanized antibody sequence
EVQLVQSGAEVKKPGESLKISCQAFGYGFINYLIEWIRQMPGQGLEWIGLINPGSDYTNYNENFKGQATISADKSSSTAYLQWSSLKASDTAMYFCARRFGYYGSGNYFDYWGQGTMVTVSS

SEQ ID NO: 51 - protein, 122 aa, Artificial sequence
  Humanized antibody sequence
EVQLVQSGAEVKKPGESLKISCQAFGYGFINYLIEWVRQMPGQGLEWIGLINPGSDYTNYNENFKGQVTISADKSSSTAYLQWSSLKASDTAMYFCARRFGYYGSGNYFDYWGQGTMVTVSS

SEQ ID NO: 52 - protein, 122 aa, Artificial sequence
  Humanized antibody sequence
EVQLVQSGAEVKKPGESLKISCQSFGYGFINYLIEWVRQMPGQGLEWIGLINPGSDYTNYNENFKGQATLSADKSSSTAYLQWSSLKASDTAMYFCARRFGYYGSGNYFDYWGQGTMVTVSS

SEQ ID NO: 53 - protein, 122 aa, Artificial sequence
  Humanized antibody sequence
EVQLVQSGAEVKKPGESLKISCQSFGYGFINYLIEWVRQMPGQGLEWIGLINPGSDYTNYNENFKGQVTISADKSSSTAYLQWSSLKASDTAMYFCARRFGYYGSGNYFDYWGQGTMVTVSS

SEQ ID NO: 54 - protein, 122 aa, Artificial sequence
  Humanized antibody sequence
EVQLVQSGAEVKKPGESLKISCQAFGYIFINYLIEWVRQMPGQGLEWIGLINPGSDYTNYNENFKGQVTISADKSSSTAYLQWSSLKASDTAMYFCARRFGYYGSGNYFDYWGQGTMVTVSS

SEQ ID NO: 55 - protein, 122 aa, Artificial sequence
  Humanized antibody sequence
EVQLVQSGAEVKKPGESLKISCQAFGYGFINYLIEWIRQMPGQGLEWIGLINPGSDYTNYNENFKGQVTISADKSSSTAYLQWSSLKASDTAMYFCARRFGYYGSGNYFDYWGQGTMVTVSS

SEQ ID NO: 56 - protein, 122 aa, Artificial sequence
  Humanized antibody sequence
EVQLVQSGAEVKKPGESLKISCQAFGYGFINYLIEWIRQMPGQGLEWIGAINPGSDYTNYNENFKGQATLSADKSSSTAYLQWSSLKASDTAMYFCARRFGYYGSGNYFDYWGQGTMVTVSS

SEQ ID NO: 57 - protein, 122 aa, Artificial sequence
  Humanized antibody sequence
EVQLVQSGAEVKKPGESLKISCQAFGYGFINYLIEWIRQMPGQGLEWIGLIYPDSGYINYNENFKGQATLSADKSSSTAYLQWSSLKASDTAMYFCARRFAYYGSGYYFDYWGQGTMVTVSS

SEQ ID NO: 58 - protein, 122 aa, Artificial sequence
  Humanized antibody sequence
EVQLVQSGAEVKKPGESLKISCQAFGYAFTNYLIEWVRQMPGQGLEWMGLIYPDSGYINYNENFKGQVTISADKSSSTAYLQWSSLKASDTAMYFCARRFAYYGSGYYFDYWGQGTMVTVSS

SEQ ID NO: 59 - protein, 122 aa, Artificial sequence
  Humanized antibody sequence
EVQLVQSGAEVKKPGESLKISCQAFGYAFTNYLIEWVRQMPGQGLEWIGLIYPDSGYINYNENFKGQATLSADKSSSTAYLQWSSLKASDTAMYFCARRFAYYGSGYYFDYWGQGTMVTVSS

SEQ ID NO: 60 - protein, 122 aa, Artificial sequence
  Humanized antibody sequence
EVQLVQSGAEVKKPGESLKISCQAFGDAFTNYLIEWVRQMPGQGLEWMGLIYPDSGYINYNENFKGQVTISADRSSSTAYLQWSSLKASDTAMYFCARRFAYYGSGYYFDYWGQGTMVTVSS

SEQ ID NO: 61 - protein, 122 aa, Artificial sequence
  Humanized antibody sequence
EVQLVQSGAEVKKPGESLKISCQAFGDAFTNYLIEWVRQMPGQGLEWIGLIYPDSGYINYNENFKGQATLSADRSSSTAYLQWSSLKASDTAMYFCARRFAYYGSGYYFDYWGQGTMVTVSS

(19) United States
(12) Patent Application Publication
(10) Pub. No.: US 2016/0075775 A1
(43) Pub. Date: Mar. 17, 2016

(54) TREATMENT OF TRAUMATIC BRAIN INJURY USING ANTIBODIES TO LYSOPHOSPHATIDIC ACID

(71) Applicant: Lpath, Inc., San Diego, CA (US)

(72) Inventors: Alice Marie PEBAY, Melbourne (AU); Ann Maree TURNLEY, Box Hill (AU); Roger A. SABBADINI, Bend, OR (US)

(21) Appl. No.: 14/953,329

(22) Filed: Nov. 28, 2015

Related U.S. Application Data
(63) Continuation-in-part of application No. 13/545,972, filed on Jul. 10, 2012.

(51) Int. Cl. C07K 16/18

(52) U.S. Cl. CPC C07K 16/18; C07K 2317/24; C07K 2317/76; A61K 2039/505

(57) ABSTRACT

Methods are provided for treating neurotrauma, for example, traumatic brain injury (TBI), using antibodies and antibody fragments that bind lysophosphatidic acid (LPA). Such treatment may result in functional locomotor recovery in subjects so treated, as well as reducing the size of a brain infarct in subjects having or suspected of having sustained neurotrauma such a TBI.

### RELATED APPLICATIONS

This patent application claims the benefit of and priority to U.S. non-provisional patent application Ser. No. 13/545,972, filed on 10 Jul. 2012 (attorney docket no. LPT-3270-CP2), which is hereby incorporated by reference in its entirety for any and all purposes.

### TECHNICAL FIELD

The present invention relates to methods for treating neurotrauma, including spinal cord injury (SCI) and traumatic brain injury (TBI), using antibodies that bind lysophosphatidic acid (LPA).

### SEQUENCE LISTING

The instant application contains a Sequence Listing submitted via the Electronic Filing System on Nov. 28, 2015 and, is hereby incorporated by reference in its entirety. Said ASCII copy, created on Nov. 25, 2015, is named LPT3270CP3.txt, and is 46,544 bytes in size.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art or even particularly relevant to the presently claimed invention.

### 2. Background

A. Neuronal Differentiation and the Role of LPA

Neural stem cells (NSC) are found in areas of neurogenesis in the central nervous system (CNS) and can migrate to sites of neural injury. Thus, NSC are under study with the goal of replacing neurons and restoring connections in a neurodegenerative environment. Dottori, et al. (2008), “Lysophosphatidic Acid Inhibits Neuronal Differentiation of Neural Stem/Progenitor Cells Derived from Human Embryonic Stem Cells.” Stem Cells 26: 1146-1154.

Following injury, hemorrhage or trauma to the nervous system, levels of LPA within the nervous system are believed to reach high levels. Dottori, et al. (ibid) have shown that LPA levels equivalent to those reached after injury can inhibit neuronal differentiation of human NSC, suggesting that high levels of LPA within the CNS following injury might inhibit differentiation of NSC into neurons, thus inhibiting endogenous neuronal regeneration. Modulating LPA signaling may thus have a significant impact in nervous system injury, allowing new potential therapeutic approaches.

B. LPA and Other Lysolipids

Lysolipids are low molecular weight lipids that contain a polar head group and a single hydrocarbon backbone, due to the absence of an acyl group at one or both possible positions of acylation. Relative to the polar head group at sn-3, the hydrocarbon chain can be at the sn-2 and/or sn-1 position(s) (the term “lyso,” which originally related to hemolysis, has been redefined by IUPAC to refer to deacylation). See “Nomenclature of Lipids, www.chem.qmul.ac.uk/iupac/lipid/lip1n2.html. These lipids are representative of signaling, bioactive lipids, and their biologic and medical importance highlight what can be achieved by targeting lipid signaling molecules for therapeutic, diagnostic/prognostic, or research purposes (Gardell, et al. (2006), Trends in Molecular Medicine, 12: 65-75). Two particular examples of medically important lysolipids are LPA (glycerol backbone) and S1P (sphingoid backbone). Other lysolipids include sphingosine, lysophosphatidylcholine (LPC), sphingosylphosphorylcholine (lysosphingomyelin), ceramide, ceramide-1-phosphate, sphinganine (dihydrosphingosine), dihydrosphingosine-1-phosphate and N-acetyl-ceramide-1-phosphate. In contrast, the plasmalogens, which contain an O-alkyl (—O—CH₂—) or O-alkenyl ether at the C-1 (sn1) and an acyl at C-2, are excluded from the lysolipid genus. The structures of selected LPAs, S1P, and dihydro S1P are presented below.

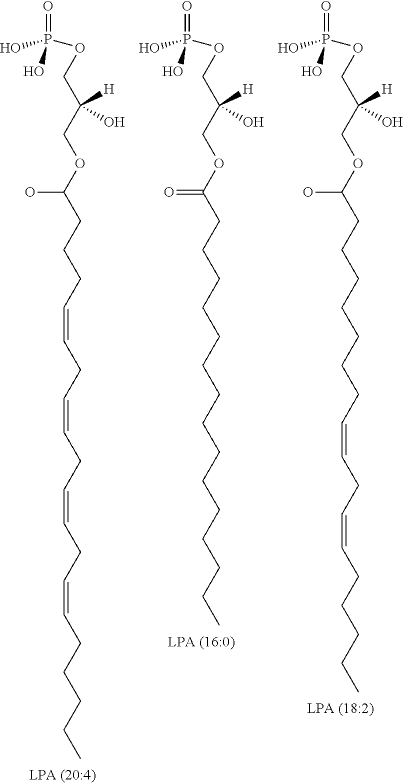

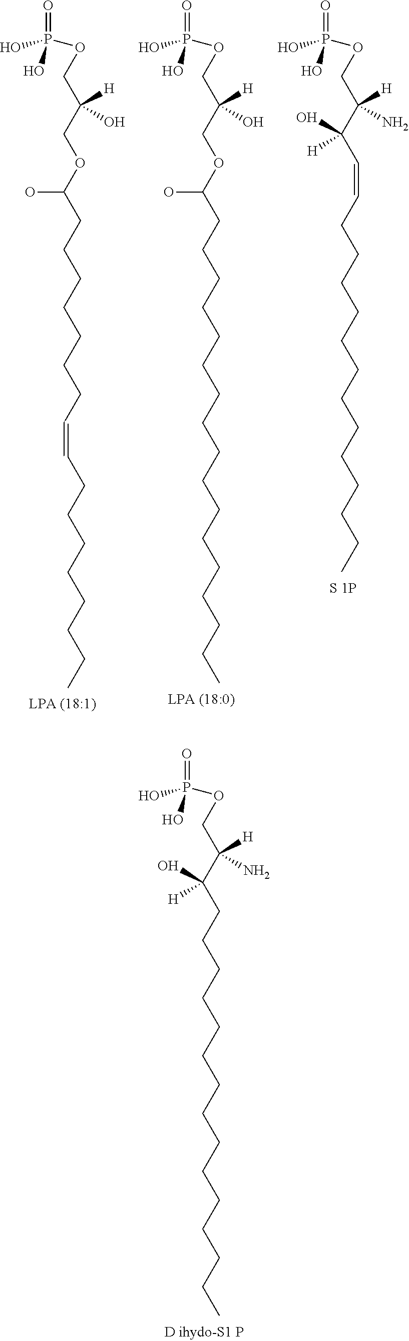

LPA is not a single molecular entity but a collection of endogenous structural variants with fatty acids of varied lengths and degrees of saturation (Fujiwara, et al. (2005), J Biol Chem 280: 35038-35050). The structural backbone of the LPAs is derived from glycerol-based phospholipids such as phosphatidylcholine (PC) or phosphatidic acid (PA). In the case of lysosphingolipids such as S1P, the fatty acid of the ceramide backbone at sn-2 is missing. The structural backbone of S1P, dihydro S1P (DHS1P) and sphingosylphosphorylcholine (SPC) is based on sphingosine, which is derived from sphingomyelin.

LPA and S1P are bioactive lipids (signaling lipids) that regulate various cellular signaling pathways by binding to the same class of multiple transmembrane domain G protein-coupled (GPCR) receptors (Chun J, Rosen H (2006), Current Pharm Des 12: 161-171, and Moolenaar, W H (1999), Experimental Cell Research 253: 230-238). The S1P receptors are designated as S1P₁, S1P₂, S1P₃, S1P₄ and S1P₅ (formerly EDG-1, EDG-5/AGR16, EDG-3, EDG-6, and EDG-8) and the LPA receptors designated as LPA₁, LPA₂, LPA₃ (formerly, EDG-2, EDG-4, and EDG-7). A fourth LPA receptor of this family has been identified for LPA (LPA₄), and other putative receptors for these lysophospholipids have also been reported.

LPA have long been known as precursors of phospholipid biosynthesis in both eukaryotic and prokaryotic cells, but LPA have emerged only recently as signaling molecules that are rapidly produced and released by activated cells, notably platelets, to influence target cells by acting on specific cell-surface receptor (see, e.g., Moolenaar, et al. (2004), BioEssays 26: 870-881, and van Leewen, et al. (2003), Biochem Soc Trans 31: 1209-1212). Besides being synthesized and processed to more complex phospholipids in the endoplasmic reticulum, LPA can be generated through the hydrolysis of pre-existing phospholipids following cell activation; for example, the sn-2 position is commonly missing a fatty acid residue due to deacylation, leaving only the sn-1 hydroxyl esterified to a fatty acid. Moreover, a key enzyme in the production of LPA, autotoxin (lysoPLD/NPP2), may be the product of an oncogene, as many tumor types up-regulate autotoxin (Brindley, D. (2004), J Cell Biochem 92: 900-12). The concentrations of LPA in human plasma and serum have been reported, including determinations made using a sensitive and specific LC/MS procedure (Baker, et al. (2001), Anal Biochem 292: 287-295). For example, in freshly prepared human serum allowed to sit at 25° C. for one hour, LPA concentrations have been estimated to be approximately 1.2 mM, with the LPA analogs 16:0, 18:1, 18:2, and 20:4 being the predominant species. Similarly, in freshly prepared human plasma allowed to sit at 25° C. for one hour, LPA concentrations have been estimated to be approximately 0.7 mM, with 18:1 and 18:2 LPA being the predominant species.

LPA influences a wide range of biological responses, ranging from induction of cell proliferation, stimulation of cell migration and neurite retraction, gap junction closure, and even slime mold chemotaxis (Goetzl, et al. (2002), Scientific World Journal 2: 324-338). The body of knowledge about the biology of LPA continues to grow as more and more cellular systems are tested for LPA responsiveness. For instance, it is now known that, in addition to stimulating cell growth and proliferation, LPA promote cellular tension and cell-surface fibronectin binding, which are important events in wound repair and regeneration (Moolenaar, et al. (2004), BioEssays 26: 870-881). Recently, anti-apoptotic activity has also been ascribed to LPA, and it has recently been reported that peroxisome proliferation receptor gamma is a receptor/target for LPA (Simon, et al. (2005), J Biol Chem 280: 14656-14662).

LPA has proven to be a difficult target for antibody production, although there has been a report in the scientific literature of the production of polyclonal murine antibodies against LPA (Chen, et al. (2000), Med Chem Lett 10: 1691-3).

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “antibody” (“Ab”) or “immunoglobulin” (Ig) refers to any form of a peptide, polypeptide derived from, modeled after, or encoded by an immunoglobulin gene, or fragment thereof, that is capable of binding an antigen or epitope. See, e.g., Immunobiology, Fifth Edition, C. A. Janeway, P. Travers, M., Walport, M. J. Shlomchiked., ed. Garland Publishing (2001).

An “antibody derivative” is an immune-derived moiety, i.e., a molecule that is derived from a first or parent antibody. This comprehends, for example, antibody fragments, antibody variants, chimeric antibodies, humanized antibodies, multivalent antibodies, antibody conjugates, and the like, which retain a desired level of binding activity for the target antigen (here, LPA).

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen binding ability, and include Fab, Fab′, F(ab′)2, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions (or “complementarity determining regions” or “CDRs”) of each heavy and light chain variable domain interact to define an antigen-binding site on the surface of the V_{H}-V_{L} dimer. Collectively, the six hypervariable (CDR) regions confer antigen-binding specificity to the antibody. However, even a single variable domain (or half of an Fv comprising only three hypervariable regions specific for an antigen) has the ability to recognize and bind antigen, although typically at a lower affinity than the entire binding site. “Single-chain Fv” or “sFv” antibody fragments comprise the V_{H} and V_{L} antibody domains, wherein those domains are present in a single polypeptide chain (typically as a fusion protein). Generally, the Fv polypeptide further comprises a polypeptide linker between the V_{H} and V_{L} domains that enables the sFv to form the desired structure for antigen binding. For a review of sFv molecules and techniques, see, e.g., Pluckthun in “The Pharmacology of Monoclonal Antibodies,” vol. 113, Rosenburg and Moore eds., Springer-Verlag, New York, pp. 269-315 (1994).

An Fab fragment also contains the constant domain of the light chain and the first constant domain (CH1) of the heavy chain. Fab′ fragments differ from Fab fragments by the addition of a few residues at the carboxyl terminus of the heavy chain CH1 domain, and generally include one or more cysteine(s) from the antibody hinge region. Fab′-SH is the designation herein for an Fab′ fragment in which the cysteine residue(s) of the constant domains bear a free thiol group. F(ab′)2 antibody fragments originally were produced as pairs of Fab′ fragments having hinge cysteines between them. Other chemical couplings of antibody fragments are also known and within the scope of the instant invention.

An “antibody variant” refers herein to a molecule that differs in amino acid sequence from a native or parent antibody (e.g., a murine monoclonal anti-LPA antibody) amino acid sequence by virtue of the addition, deletion, and/or substitution of one or more amino acid residue(s) in the antibody sequence and that retains at least one desired activity of the parent anti-binding antibody. Desired activities can include the ability to bind the antigen specifically, the ability to inhibit proliferation in vitro, the ability to inhibit angiogenesis in vivo, and the ability to alter cytokine profile in vitro. The amino acid change(s) in an antibody variant may be within a variable region or a constant region of a light chain and/or a heavy chain, including in the Fc region, the Fab region, the CH₁ domain, the CH₂ domain, the CH₃ domain, and the hinge region. In one embodiment, the variant comprises one or more amino acid substitution(s) in one or more hypervariable region(s) (CDR(s)) of the parent antibody. For example, the variant may comprise at least one, e.g., from about one to about ten, and preferably from about two to about five, substitutions in one or more hypervariable regions (CDRs) of the parent antibody. Ordinarily, the variant will have an amino acid sequence having at least about 75% amino acid sequence identity with the parent antibody heavy or light chain variable domain sequences, more preferably at least about 65%, more preferably at least about 80%, more preferably at least about 85%, more preferably at least about 90%, and most preferably at least about 95%. Identity or homology with respect to aminoa acid sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind LPA and preferably has desired activities that are superior to those of the parent antibody. For example, the variant may have a stronger binding affinity, enhanced ability to reduce angiogenesis and/or halt tumor progression, exhibit reduced aggregation during purification, have superior pharmacokinetic and/or pharmacodynamics properties, etc. To analyze such desired properties (for example, reduced immunogenicity, longer half-life, enhanced stability, enhanced potency), one should compare a Fab form of the variant to a Fab form of the parent antibody or a full length form of the variant to a full length form of the parent antibody, for example, since it has been found that the format of the anti-LPA antibody impacts its activity in the biological activity assays described herein. A variant antibody of particular interest herein can be one that displays at least about 10 fold, preferably at least about 5%, 25%, 50%, 100%, 200%, or 500%, or more of at least one desired activity. Preferred variants are those one that have superior biophysical properties as measured in vitro or superior biological activities as measured in vitro or in vivo when compared to the parent antibody.

The term “antigen” refers to a molecule that is recognized and bound by an antibody or antibody derivative that binds to the antigen.

An “epitope” or “antigenic determinant” refers to that portion of an antigen that reacts with an antibody or antigen-binding portion derived from an antibody.

An “anti-LPA antibody” refers to any antibody or antibody-derived molecule that binds lysophosphatidic acid. The terms “anti-LPA antibody,” “antibody that binds LPA”, and “antibody reactive with LPA” are interchangeable.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes.

The term “biologically active,” in the context of an antibody or antibody fragment or variant, refers to an antibody or antibody fragment or antibody variant that is capable of binding the desired epitope and in some way exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body that has a particular molecular feature that makes it useful for measuring the susceptibility to or progress of disease or the effects of treatment.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other suitable classes and examples of carriers are known in the art. These, as well as later discovered or invented naturally occurring or synthetic carriers, can be adapted for application in accordance with the disclosure herein.

The term “chimeric” antibody (or immunoglobulin) refers to a molecule comprising a heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., Cabilly, et al., infra; Morrison, et al. (1984), Proc. Natl. Acad. Sci. U.S.A. 81:6851).

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting therapeutic agent and an anti-lipid antibody (e.g., an anti-LPA antibody). Alternatively, a combination therapy may involve the administration of an anti-lipid antibody and/or one or more therapeutic agents, alone or together with the delivery of another treatment, such as radiation therapy and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more therapeutic agents are combined with, for example, radiation and/or surgery, the drug(s) may be delivered before or after surgery or radiation treatment.

“Effective concentration” refers to the absolute, relative, and/or available concentration and/or activity, for example, of certain undesired bioactive lipids. In other words, the effective concentration of a bioactive lipid is the amount of lipid available, and able, to perform its biological function. As described herein, an anti-LPA antibody or LPA-binding antibody fragment, is able to reduce the effective concentration of the lipid by binding to the lipid and rendering it unable to perform its biological function. In the foregoing example, the LPA itself is still present (i.e., it is not degraded by the antibody, in other words) but can no longer bind its receptor or other targets to cause a downstream effect, so “effective concentration” rather than absolute concentration is the appropriate measurement. Methods and assays exist for directly and/or indirectly measuring the effective concentration of bioactive lipids, particularly LPA.

A “fully human antibody” can refer to an antibody produced in a genetically engineered (i.e., transgenic) mouse (e.g., from Medarex) that, when presented with an immunogen, can produce a human antibody that does not necessarily require CDR grafting. Such antibodies are fully human (100% human protein sequences) from animals such as mice in which the non-human antibody genes are suppressed and replaced with human antibody genes. The applicants believe that antibodies useful in practicing the invention can be generated against bioactive lipids when presented to such genetically engineered mice or other animals able to produce human frameworks for the relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibodies, antibody fragment(s), or other immune-derived binding moieties to one or more molecule(s), directly or via a linker moiety.

“Humanized” forms of non-human (e.g., murine) antibodies are chimeric antibodies that contain minimal sequence derived from non-human immunoglobulins. Or, looked at another way, a humanized antibody is a human antibody that also contains selected sequences, e.g., CDRs, from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibodies) in which residues from a complementary-determining region (CDR) of the recipient molecule are replaced by residues from a CDR of a non-human species (donor antibody) such as mouse, rat, camel, bovine, goat, or rabbit having the desired properties. In some instances, one or more framework region (FR) residues of a human immunoglobulin are replaced by corresponding non-human residues. The CDRs can be placed into any of a variety of frameworks as long as a desired level of antigen binding is retained, or is restored or enhanced by techniques.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European pat. no. 0,125,023 B1; Boss, et al., U.S. Pat. No. 4,816,397; Boss, et al., European pat. no. 0,120,694 B1; Neuberger, et al., WO 86/01533; Neuberger, et al., European pat. no. 0,194,276 B1; Winter, U.S. Pat. No. 5,225,539; Winter, European pat. no. 0,239,400 B1; Padlan, et al., European patent application no. 0,519,596 A1; Queen, et al. (1989), Proc. Nat'l Acad. Sci. USA, 86:10029-10033). For further details, see Jones, et al., Nature 321:522-525 (1986); Reichmann, et al., Nature 332:323-329 (1988); and Presta, Curr. Op. Struct. Biol. 2:593-596 (1992), and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include but are not limited to disorders of excessive scarring (i.e., fibrosis) such as age-related macular degeneration, cardiac remodeling and failure associated with myocardial infarction, excessive wound healing such as commonly occurs as a consequence of surgery or injury, keloids, and fibroid tumors and stenting.

An “immunogen” is a molecule capable of inducing a specific immune response, particularly an antibody response in an animal to whom the immunogen has been administered. The immunogen used to generate the antibodies described herein is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus, the immunogen may also be used as a detection reagent. Alternatively, the derivatized bioactive lipid conjugate used as capture material may have a different linker and/or carrier moiety from that in the immunogen.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” antibody is one that has been identified and separated and/or recovered from a component of its natural environment or the environment in which it was produced (e.g., a recombinant expression system). Contaminant components of its natural or other synthetic environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody or antibody derivatives will be prepared by at least one purification step.

The word “label” when used herein refers to a detectable compound or composition, such as one that is conjugated directly or indirectly to the antibody. The label may be detectable by itself (e.g., radioisotope labels or fluorescent labels) or, in the case of an enzymatic label, may catalyze chemical alteration of a substrate compound or composition that is detectable.

In the context of this disclosure, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The term “metabolites” refers to compounds from which LPAs are made, as well as those that result from the degradation of LPAs; that is, compounds that are involved in the lysophospholipid metabolic pathways. The term “metabolic precursors” may be used to refer to compounds from which sphingolipids are made.

The term “monoclonal antibody” (mAb) as used herein refers to a population of substantially homogeneous antibodies, or to said population of antibodies. The individual antibodies comprising the population are essentially identical in terms of amino acid sequences, except for possible naturally occurring mutations or post-translational modifications that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being one of a substantially homogeneous population of antibodies, and is not to be construed as requiring production of the antibody by any particular method. For example, monoclonal antibodies may be made by the hybridoma method first described by Kohler, et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson, et al., Nature 352:624-628 (1991) and Marks, et al., J. Mol. Biol. 222:581-597 (1991), for example, or by other methods known in the art. The monoclonal antibodies herein specifically include chimeric antibodies (or antigen-binding fragments of such antibodies) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison, et al., Proc. Natl. Acad. Sci. USA 81:6851-6855 (1984)).

“Monotherapy” refers to a treatment regimen based on the delivery of one therapeutically effective compound, whether administered as a single dose or several doses over time.

“Neural” means pertaining to nerves. Nerves are bundles of fibers made up of neurons.

“Neural stem cells” (NSCs) are the self-renewing, multipotent cells that differentiate into the main phenotypes of the nervous system. NSCs give rise to glial and neuronal cells. Neuronal stem cells give rise to neuronal cells. Neural progenitor cells (NPCs) are the progeny of stem cell division that normally undergo a limited number of replication cycles in vivo.

“Neuron” refers to an excitable cell type in the nervous system that processes and transmits information by electrochemical signalling. Neurons are the core components of the CNS (central nervous system, comprised of the brain and spinal cord) and the peripheral nerves. “Neuronal” means “pertaining to neurons.”

“Neuronal differentiation” is the conversion of neural stem cells toward the mature cell types of the nervous system, such as neurons, astrocytes, etc. Such differentiation occurs in vivo but can be caused to occur in vitro in model systems such as neurospheres. Differentiation may be a multistep or multistage process and thus multiple phases or steps of differentiation can be studied in vitro.

A “parent” antibody is one having an amino acid sequence used for the preparation of an antibody derivative or variant. A parent antibody may be a native antibody or may already be a variant, e.g., a chimeric antibody. For example, the parent antibody may be a humanized or human antibody, and an antibody derivative of such an antibody would, for example, include a Fab fragment derived therefrom.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci. 66, 1-19).

A “plurality” means more than one.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol, and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

The term “species” is used herein in various contexts, e.g., a particular chemical or molecular species (e.g., a particular chemotherapeutic agent or anti-LPA antibody). In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus, an antibody is commonly said to “bind” (or “specifically bind”) or be “reactive with” (or “specifically reactive with”), or, equivalently, “reactive against” (or “specifically reactive against”) the epitope of its target antigen. Antibodies are commonly described in the art as being “against” or “to” their antigens as shorthand for antibody binding to the antigen. Thus, an “antibody that binds LPA,” an “antibody reactive against LPA,” an “antibody reactive with LPA,” an “antibody to LPA,” and an “anti-LPA antibody” all have the same meaning. Antibody molecules can be tested for specificity of binding by comparing binding to the desired antigen to binding to unrelated antigen or analogue antigen or antigen mixture under a given set of conditions. Preferably, an antibody lacks significant binding to unrelated antigens, or even analogs of the target antigen.

A “subject” or “patient” refers to an animal in need of treatment that can be effected by antibodies or antigen-binding antibody fragments described herein. Animals that can be treated include vertebrates, with mammals such as bovine, canine, equine, feline, ovine, porcine, and primate (including humans and non-human primates) animals being particularly preferred examples.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as used for cancer, cardiovascular agents, immunomodulatory agents, agents that are used to treat neurodegenerative disorders, ophthalmic drugs, etc.

A “therapeutically effective amount” (or “effective amount”) refers to an amount of an active ingredient, e.g., an agent according to the disclosure, sufficient to effect treatment when administered to a subject in need of such treatment. Accordingly, what constitutes a therapeutically effective amount of a composition may be readily determined by one of ordinary skill in the art. For example, in the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of that active ingredient when administered as a monotherapy (i.e., a therapeutic regimen that employs only one chemical entity as the active ingredient).

As used herein, the terms “therapy” and “therapeutic” encompasses the full spectrum of prevention and/or treatments for a disease, disorder, or physical trauma. A “therapeutic” agent may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments.

The term “treatment” or “treating” means any therapy of or for a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. For example, in traumatic brain injury (TBI), the initial injury is followed by a second phase of inflammation and neurodegeneration, as is described below. “Treatment” of TBI may, therefore, include, e.g., neuroprotection (such as minimization of a brain contusion), reduction or inhibition of inflammation, reduction or inhibition of neurodegeneration, and improved functional recovery, such as locomotor improvement. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys, and other protein-based therapeutics.

The term “variable” region (of an antibody) comprises framework and complementarity regions or CDRs (otherwise known as hypervariable regions) refers to certain portions of the variable domains that differ extensively in sequence among antibodies and are used in the binding and specificity of each particular antibody for its particular antigen. However, the variability is not evenly distributed throughout the variable domains of antibodies. It is concentrated in three segments called hypervariable regions (CDRs) both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example, residues 24-34 (L1), 50-56 (L2) and 89-97 (L3) in the light chain variable domain and 31-35 (H1), 50-65 (H2) and 95-102 (H3) in the heavy chain variable domain; Kabat, et al., Sequences of Proteins of Immunological Interest, 5th Ed. Public Health Service, National Institutes of Health, Bethesda, Md. (1991)) and/or those residues from a “hypervariable loop” (for example residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined. The hypervariable regions in each chain are held together in close proximity by the FRs and, with the hypervariable regions from the other chain, contribute to the formation of the antigen-binding site of antibodies (see Kabat, et al., above, pages 647-669). Thus the uniqueness of an antibody for binding its antigen comes from the CDRs (hypervariable regions) and their arrangement in space, rather than the particular framework that holds them there. The CDRs can be placed into any of a variety of frameworks as long as a desired level of antigen binding is retained.

The constant domains are not involved directly in binding an antibody to an antigen, but exhibit various effector functions, such as participation of the antibody in antibody-dependent cellular toxicity.

### SUMMARY OF THE INVENTION

The present invention concerns methods for treating neurotrauma (e.g., traumatic brain injury, stroke, brain or spinal cord hemorrhage, brain infarct, and spinal cord injury) in a subject, particularly a human subject. Such methods involve treating the subject with a specified antibody or antibody fragment that binds lysophosphatidic acid. Also provided are methods for reducing the size of a brain infarct in subjects known or suspected to have sustained neurotrauma, as well methods for increasing locomotor recovery in such subjects.

The methods of the invention each involve administering a therapeutically effective amount of an antibody, or fragment thereof, that binds lysophosphatidic acid (LPA). Such antibodies and antibody fragments may be a monoclonal antibodies, as well as variants or derivatives thereof. Humanized anti-LPA antibodies, and LPA-binding fragments of such antibodies, are preferred.

In some embodiments of the methods of the invention, the antibody or LPA-binding fragment thereof comprises or consists essentially of at least one, and preferably two, light chain variable domain(s) comprising or consisting essentially of an amino acid sequence: EVQLVQSGAEVKKPGESLKISCQAFGDAFTNYLIEWVRQMPGQGLEWIGLIYPDSGYINYNENFKGQATLSADRSSSTAY LQWSSLKASDTAMYFCARRFAYYGSGYYFDYWGQGTMVTVSS (SEQ ID NO: 61) and at least one, and preferably two, light chain variable domain(s) comprising or consisting essentially of an amino acid sequence: DVVMTQTPLSLPVTPGEPASISCRSSQSLLKTNGNTYLHWYLQKPGQSPKLLIFKVSNRFSGVPDRFSGSGSGTDFTLKI SRVEAEDVGVYFCSQSTHFPFTFGQGTKLEIK (SEQ ID NO: 42).

A particularly preferred embodiment of the invention utilizes the humanized anti-LP antibody designated herein as “LT3114”.

These and other aspects and embodiments are discussed in greater detail in the sections that follow. As those in the art will appreciate, the following description describes certain preferred embodiments in detail, and is thus only representative and does not depict the actual scope of the invention. It is understood that the invention is not limited to the particular molecules, systems, and methodologies described, as these may vary. It is also to be understood that the terminology used herein is for the purpose of describing particular embodiments only, and is not intended to limit the scope of the invention defined by the appended claims.

### BRIEF DESCRIPTION OF THE DRAWINGS

This application contains several figures executed in color. Copies of this application with color drawings will be provided upon request and payment of the necessary fee. A brief summary of the figures is provided below.

FIGS. 1A-1D are a series of four bar graphs showing that LPA inhibits neurosphere formation and neuronal differentiation of hNS/PC. Specifically, FIG. 1A plots eurosphere formation by NS/PC cultivated for 5 days with or without LPA (10 μM unless otherwise mentioned) and/or Y27632 (1 μM). FIG. 1B is a plot showing cell proliferation by Ki67 staining and apoptosis by TUNEL of neurospheres treated or not with LPA (10 μM) and/or Y27632 (1 μM) for 5 days. FIG. 1C is a plot of data for neuron-forming neurospheres in the absence or presence of LPA (1 μM) and/or anti-LPA mAb B3 (1 mg/ml) for 3 days. FIG. 1D is a plot representing neurosphere formation. Data are means±SEM, n≧3 independent experiments. **p<0.01, ***p<0.001 by one-way ANOVA, T-test.

FIGS. 2A and 2B are micrographs showing mouse brains after cortical injury. Specifically, FIG. 2A is a micrograph showing a mouse brain after cortical injury with an area of hemorrhage as typically seen after TBI in the cortical impact model described herein, and FIG. 2B is a micrograph showing a mouse brain after cortical injury TBI in the same model depicted in FIG. 2A, but treated with anti-LPA antibody. When the micrographs in FIGS. 2A and 2B are compared, the hemorrhage normally observed in this model is seen to be greatly reduced.

FIG. 3A shows a series of photographs of 12 mouse brains following TBI that demonstrate that an anti-LPA antibody is protective in a mouse model of traumatic brain injury. The 6 brains shown in the photographs in the top panel (Con) were from mice that received no antibody treatment prior to TBI. The 6 brains in the lower panel (Mab) were from mice that received the anti-LPA antibody B3, 0.5 mg/mouse i.v., prior to the application of a single impact injury (1.5 mm depth). Mice were taken down 24 hrs following injury.

FIG. 3B is a plot showing that anti-LPA antibody is protective in a mouse model of traumatic brain injury. FIG. 3B also shows histological quantitation of the infarct volumes in the animals studied. As shown, the decrease in infarct size in anti-LPA antibody-treated mice compared to controls is statistically significant.

FIG. 4 is a scatter plot showing that anti-LPA mAb intervention treatment significantly reduces neurotrauma following TBI. Mice were subjected to TBI using Controlled Cortical Impact (CCI) and treated with either control mAb or B3 given as single i.v. dose of 25 mg/kg 30 min after injury. Data were obtained 2 days after injury and infarct size for each animal was quantified histologically. Data are means±SEM, n=8 animals per group in from two independent, blinded studies.

FIGS. 5A and 5B show that anti-LPA mAb intervention treatment significantly reduces neurotrauma following TBI. Mice were subjected to TBI using Controlled Cortical Impact (CCI) and treated with either control mAb or B3 given as single i.v. dose of 25 mg/kg 30 min after injury. Data were obtained seven days after injury. Specifically, FIG. 5A is a graph showing anti-LPA mAb intervention treatment significantly reduces neurotrauma following TBI. This graph shows histological quantification of infarct size assessed by MRI seven days post-injury. FIG. 5B is a pair of photographs showing representative MRI images of mouse brains following TBI and subsequent treatment with anti-LPA antibody or isotype control antibody. Data are means±SEM, n=8 animals per group in from two independent, blinded studies. *p<0.05.

FIGS. 6A and 6B show that treatment with anti-LPA antibody B3 improves functional recovery following TBI. Animals were treated with anti-LPA antibody B3 or isotype control antibody and tested weekly in the grid-walking model. The number of faults in foot placement was reduced at all time points after 3 days post-injury in forelimbs after anti-LPA antibody (B3) treatment compared to control antibody treatment. Specifically, FIG. 6A shows the number of faults in forelimb foot placement in treated versus control animals, and FIG. 6B shows the number of faults in hindlimb foot placement in treated versus control animals.

FIGS. 7A and 7B show that post-injury (2 hr) treatment with anti-LPA antibody protects mice from long-term functional/behavioral consequences in the CCI model of TBI. Total faults were measured of 50 steps for front limbs and 50 steps for hindlimbs. Data are shown as median and 95% confidence intervals. P-values indicate significant difference between anti-LPA and IgG treatment groups and were obtained by bootstrapping means around the confidence intervals in R; P-values *P<0.05, **P<0.001, ***P<0.0001. Specifically, FIG. 7A is a bar graph showing the total number of front limb faults out of 50 steps, and FIG. 7B is a bar graph showing the total number of hind limb faults out of 50 steps.

FIGS. 8A and 8B show that anti-LPA mAb (B3) reduces glial scar following SCI, as measured by immunostaining at the injury site of mice spinal cords, 2 weeks following SCI. Mice received or not anti-LPA mAb (B3, 0.5 mg/mouse) subcutaneously twice a week for two weeks, starting just after SCI. Specifically, FIG. 8A shows that anti-LPA antibody B3 treatment reduced the amount of reactive astrocytes (GFAP and CSPG cells), and FIG. 8B shows that anti-LPA antibody B3 treatment increased the amount of neurons (NeuN) close to the lesion site.

FIGS. 9A and 9B show that treatment with anti-LPA antibody B3 improves functional recovery following SCI. mBBB score and grid walking test were measured up to 5 weeks post-SCI. In the study, mice treated with antibody B3 (n=7) were compared to mice treated with isotype control antibody (con; n=8), given for two weeks following SCI. Data in FIGS. 9A and 9B are mean±SEM; *p<0.05. Specifically, FIG. 9A is a line graph showing the mBBB open field locomotor test scores, and FIG. 9B is a line graph showing grid walking test scores.

FIG. 10 is a bar graph showing that antibody to LPA improves neuronal survival following spinal cord injury (SCI). Quantitation of number of traced neuronal cells rostral to lesion site is significantly higher in antibody treated mice compared to controls. Data are mean±SEM;**p<0.001.

FIG. 11 is a scatter plot showing total LPA levels (in μM) in CSF of patients within the first six days after TBI. Measurements were made by liquid chromatography-mass spectrometry (LC-MS).

FIG. 12 is a bar graph showing an increase (pulse) in 18:2 LPA (in μM) in CSF of patients within the first day after TBI, and a return to baseline levels by day 5 post-injury. Measurements were made by liquid chromatography-mass spectrometry (LC-MS).

FIGS. 13A-13C show the relationship between total LPA levels (in μM) in CSF over the first 36 hr post-TBI and the severity of injury using three accepted clinical scores of injury severity, the Glasgow Coma Scale (GCS, FIG. 13A), the Injury Severity Scale (ISS, FIG. 13B), and the Extended Glasgow Outcome Scale (GOSE, FIG. 13C) assessed 6 months post-injury. Specifically, FIG. 13A is a scatter plot showing the relationship between total LPA levels (in μM) in CSF over the first 36 hr post-TBI and the severity of injury using the Glasgow Coma Scale (GCS). FIG. 13B is a scatter plot showing the relationship between total LPA levels (in μM) in CSF over the first 36 hr post-TBI and the severity of injury using the Injury Severity Scale (ISS) assessed in acute phase. FIG. 13C is a scatter plot showing the relationship between total LPA levels (in μM) in CSF over the first 36 hr post-TBI and the severity of injury using the Extended Glasgow Outcome Scale (GOSE) assessed 6 months post-injury.

### DETAILED DESCRIPTION OF THE INVENTION

The present invention relates to methods for treating neurotrauma, including TBI and SCI, using antibodies to lysolipids, particularly lysophosphatidic acid (LPA), as well as methods for reducing the size of a brain infarct in subjects known or suspected to have sustained neurotrauma, as well methods for increasing locomotor recovery in such subjects.

### 1. Neurotrauma

Neurotrauma refers to injury to the CNS, whether through trauma, hemorrhage, or disease. Major types of neurotrauma include spinal cord injury (SCI), traumatic brain injury (TBI), and stroke, which may be ischemic or hemorrhagic. CNS injury is a type of injury likely to result in death or lifelong disability.

Various scoring systems are commonly used clinically to quickly and concisely evaluate and convey the severity of CNS injury. The Glasgow Coma Scale (GCS) is used to quantitate the severity of coma in a patient who has suffered a traumatic brain injury. Mental alertness varies from fully alert to lethargic and stuporous all the way to deep coma, where a patient is minimally responsive or unresponsive to external stimuli. The GCS grades the level of a subject's consciousness on a scale from 3 (worst, deep coma) to 15 (normal, alert). A Coma Score of 13 or higher indicates a mild brain injury, 9 to 12 a moderate injury, and 8 or less a severe brain injury.

The Extended Glasgow Outcome Scale (GOSE) is a practical index of outcome or recovery following head injury designed to complement the Glasgow Coma Scale. The eight levels of recovery are: 1) Dead; 2) Vegetative State; 3) Lower Severe Disability; 4) Upper Severe Disability; 5) Lower Moderate Disability; 6) Upper Moderate Disability; 7) Lower Good Recovery; and 8) Upper Good Recovery.

The Injury Severity Scale (ISS) is an anatomical scoring system that provides an overall score for patients with multiple injuries. Each injury is assigned an Abbreviated Injury Scale (AIS) score (from 1 to 6, with 1 being minor, 5 severe, and 6 an unsurvivable injury) and is allocated to one of six body regions (Head, Face, Chest, Abdomen, Extremities (including Pelvis), External). Only the highest AIS score in each body region is used. The score for each of the three most severely injured body regions is squared and added together to produce the ISS score.

a. Traumatic Brain Injury (TBI)

TBI is a disruption of function in the brain that results from a blow or jolt to the head or penetrating head injury. There are more than 1.5 million TBIs per year in the US, with 125,000 of these resulting in permanent disability. Moreover, TBI is the leading cause of military casualties in the field and is a leading source of long-term rehabilitation problems suffered by veterans. When not fatal (22% of moderate and 35% of severe TBI patients die within the first year following injury), TBI can result in permanent and severe physical, cognitive, and behavioral impairments, leaving sufferers in need of long term healthcare. Currently, there are no FDA-approved drugs targeting TBI.

TBI is heterogeneous in its causes and can be seen as a two-step event: 1) a primary injury, which can be focal or diffuse, caused by mechanical impact or other traumatic injury to the head or body, that results in primary pathological events such as hemorrhage and ischemia, tearing of tissue and axonal injuries; 2) a secondary injury such as diffuse inflammation, cell death, and gliosis, which is a consequence of the primary one. This secondary injury starts immediately after injury and can continue for weeks, and is thought to involve an active inhibition of neural stem cell activity. Collectively, these events lead to neurodegeneration.

A large fraction of TBI are mild, and thus may go undiagnosed immediately after injury. Because there is no single TBI symptom or pattern of symptoms that characterize mild TBI, no rapid diagnostic test, for example, a rapid screening test, ideally one (such as a kit described herein) that can be used in the field, an emergency room or in a rescue vehicle, has yet been developed. Undiagnosed and untreated TBI presents a risk because some signs and symptoms may be delayed from days to months after injury, and may have significant impact on the patient's physical, emotional, behavioral, social, or family status if untreated, and may result in a functional impairment. Because secondary damage from the injury continues after the initial impact, early treatment (and thus rapid diagnosis), particularly point-of-care treatment, is desirable. An ideal therapy for TBI would reduce the injury infarct size as well as limit any secondary inflammatory response(s). As is described herein, the inventors have discovered that anti-LPA antibodies provide substantial neuroprotection when given after TBI, and mitigating the inflammation and stimulating the neuroregeneration responses important for long-term positive outcomes. Without wishing to be bound to a particular theory, it is believed that anti-LPA antibody (or LPA-binding antibody fragment) treatment provides an unexpected and unique approach to limit the initial infarct, hemorrhage, and inflammation and also stimulate regenerative processes to optimize functional recovery.

An increasingly prevalent subset of TBI is blast-induced or blast TBI (bTBI). With the increasing use of explosives, including improvised explosive devices (IEDs) on battlefields around the world, bTBI among soldiers and civilians is also increasing. Such injuries are often referred to as the hallmark injury of the wars in Iraq and Afghanistan, and affect both military and civilian workers in battle zones. Blast injuries are the most common cause of TBI in U.S. soldiers in combat and are a major cause of disability among service members.

Blast injuries can result in the full spectrum of closed and penetrating TBIs (mild, moderate, and severe). Mild and moderate TBI's are more prevalent than severe injuries in the current military conflict due to the vast improvement in protective gear, leading to an increase in survivors of bTBI.

Blast injuries are defined by four potential mechanism dynamics:
Primary Blast: Atmospheric over-pressure followed by under-pressure or vacuum.
Secondary Blast: Objects placed in motion by the blast hitting the subject.
Tertiary Blast: Subject being placed in motion by the blast.
Quaternary Blast: Other injuries from the blast such as burns, crush injuries, amputations, and toxic fumes.

Blast TBIs are typically closed-head injuries and are more complex than other forms of TBI, with multiple mechanisms of injury, including shockwave transmission through the skull and sensory organs of the head. In a patient sample seen in the Department of Veterans Affairs (VA) polytrauma system, the pattern of injuries was different among those with injuries due to blasts versus other mechanisms. Injuries to the face (including eye, ear, oral, and maxillofacial), penetrating brain injuries, symptoms of posttraumatic stress, and auditory impairments are more common in blast-injured patients than in those with war injuries of other etiologies. Sayer, et al. (2008), Arch Phys Med Rehabil. Jan; 89:163-70.

b. Spinal Cord Injury (SCI)

SCI usually begins with a sudden, traumatic blow to the spine that fractures or dislocates vertebrae, or with an injury that transects the spinal cord. The damage begins at the moment of injury when the cord is directly damaged, or when surrounding bone, discs, or ligaments bruise or tear spinal cord tissue, causing destruction of axons, which are the long extensions of nerve cells that carry signals up and down the spinal cord between the brain and the rest of the body. An injury to the spinal cord can damage a few, many, or most of these axons, and the extent of the resulting paralysis and loss of sensation is variable as a result. Improved emergency care and aggressive treatment and rehabilitation can help minimize damage to the nervous system and even restore limited abilities. Surgery may be needed to relieve compression of the spinal cord and to repair fractures. The steroid drug methylprednisolone appears to reduce the damage to nerve cells if it is given within the first 8 hours after injury. In addition to paralysis and loss of sensation, SCI is often accompanied by respiratory problems (with higher levels of injury often requiring ventilator support), chronic pain and bladder and bowel dysfunction, and an increased susceptibility to heart problems.

c. Stroke

A stroke is a sudden interruption of blood flow to the brain caused by hemorrhage (bleeding) in the brain, usually caused by a ruptured blood vessel, or by a loss of blood flow (ischemia) to an area of the brain, such as may be caused by a blood clot lodging in an artery supplying blood to a portion of the brain. Ischemic strokes account for the vast majority of stroke. Strokes may cause sudden weakness, loss of sensation, or difficulty with speaking, seeing, or walking. Symptoms vary according to the location and extent of the interruption in blood flow and resulting tissue damage. Stroke is the third leading cause of death and the leading cause of serious, long-term disability in the United States. Stroke is typically determined by physical examination, particularly by imaging such as CT scan, MRI scan, etc. Stroke cannot currently be diagnosed by blood test(s). Blood tests, however, may be done to further understand the medical condition that has lead to stroke symptoms. Lumbar puncture is often performed if a stroke due to subarachnoid hemorrhage is suspected, or if other CNS conditions such as meningitis are suspected.

### 2. LPA in CNS Injury

Key components of the LPA pathway are modulated following neurotrauma. In postmortem brains of patients who died following acute closed head injury, expression of LPA receptors was upregulated compared to expression levels in postmortem brains from normal individuals. LPA₁ upregulation co-localized with astrocytes, while LPA₂ upregulation occurred on the ependymal cell layer of the lateral ventricles. Frugier, et al. (2011), Cell Mol Neurobiol 31:569-77. More recently, Crack, et al. ((27 Feb. 2014), J Neuroinflamm 11:37), have shown that LPA levels increase significantly in cerebrospinal fluid (CSF) drained to reduce intracranial pressure in patients with severe traumatic brain injury (TBI). CSF was drained daily from day of admission (day 0) to day 5, and levels of LPA were measured in the collected CSF. Total LPA levels in the CSF of brain injured patients were found to be elevated compared to those in the CSF of patients who had not sustained a brain injury. A significant and substantial elevation (from 0.05 μM in uninjured control samples to 0.270 μM in brain injured samples) occurred within the first 24 hr after injury and levels returned to basal levels by 120 hr after injury. This increase is referred to as an “LPA pulse”, and the authors reported that the 16:0 and 18:0 isoforms of LPA are predominant components of the pulse.

LPA receptors 1-3 (LPA₁₋₃) are also strongly upregulated in response to injury in the mouse. Goldshmit, et al. (2010), Cell Tissue Res. 341:23-32. Examination of LPA receptors expression in the intact uninjured spinal cord showed that LPA₁₋₃ are expressed at low but distinct levels in different areas of the spinal cord. LPA₁ is expressed in the central canal by ependymal cells, while LPA₂ is expressed in cells immediately surrounding the central canal and at low levels on some astrocytes in the grey matter. LPA₃ is expressed at low levels on motor neurons of the ventral horn and throughout the grey matter neuropil. Following SCI, LPA₁ is still expressed on a subpopulation of astrocytes near the injury site at four days following injury, although its level of expression is increased. LPA₂ is expressed by astrocytes, with an upregulation on reactive astrocytes around the lesion site by two days, and further increased by four days. LPA₃ expression remains confined to neurons but is upregulated in a small number of neurons by two days post-injury, and further increased by four days extending its expression to the neuronal processes. This upregulation is observed not only close to the lesion site, but also distal from both sides. More recently, Crack, et al. (2014, supra) also reported measured LPA levels in mouse CSF following experimental TBI in the closed cortical impact (CCI) model, which closely reproduces the closed head injury in the clinical study described above and in Example 17, below. Total LPA levels were reportedly found to increase in mice within three hours after injury, and returned to normal fourteen hours post-injury, showing a dysregulation of LPA soon after injury, as in the clinical study. In mice, the predominant isoform in this LPA pulse was 18:0 LPA.

Considering the pleiotropic effects of LPA on most neural cell types, especially on cell morphology, proliferation, and survival, together with demonstration of a localized upregulation of LPA₁₋₃ following injury, it is likely that LPA regulates essential aspects of the cellular reorganization following neural trauma by being a key player in reactive astrogliosis, neural regeneration and axonal re-growth. Data strongly suggest that neural responses to LPA stimuli are likely to significantly influence the amount of ensuing damage or repair following brain and/or spinal cord injury. Elevated levels of LPA are observed in certain pathological states including brain and spinal cord injury. LPA injections into mouse brain induce astrocyte reactivity at the site of the injury, while in the spinal cord, LPA induces neuropathic pain and demyelination. LPA can stimulate astrocytic proliferation and can promote death of hippocampal neurons. Moreover, LPA mediates microglial activation and is cytotoxic to the neuromicrovascular endothelium.

Following injury, LPA is synthesized in the mouse spinal cord in a model of sciatic nerve ligation (Ma, Uchida et al. 2010) and LPA-like activity is increased in the cerebrospinal fluid in a model of cerebral hematoma in newborn pigs (Tigyi, et al. (1995), Am J. Physiol. 268:H2048-2055; Yakubu, et al. (1997), Am J. Physiol. 273:R703-709). Normally undetectable, levels of ATX (autotaxin, an enzyme that can synthesize LPA) increase in astrocytes neighboring a lesion of the adult rat brain (Savaskan, et al. (2007), Cell Mol. Life Sci., 64:230-43). In humans, the presence of ATX in cerebrospinal fluid has been demonstrated in multiple sclerosis patients (Hammack, et al. (2004), Mult Scler. 10:245-60 and higher levels of LPA in human plasma might predict silent brain infarction (Li, et al. (2010), Int J Mol Sci. 11:3988-98). Further, in human cerebrospinal fluid from traumatic brain injury (TBI) patients (Farias, et al. (2011), J Trauma. 71:1211-8), levels of arachidonic acid, a lipid generated from the hydrolysis of phosphatidic acid into LPA and arachidonic acid, increase. Although not studied in this report, the Farias, et al. data suggest a parallel increase of LPA following TBI.

Overall, these studies indicate that LPA and its related molecules participate in different developmental events of the CNS, and increase dramatically in pathological conditions when compared to normal physiological levels.

### 3. Antibodies

Antibody molecules or immunoglobulins are large are heterotetrameric glycoprotein molecules with a molecular weight of approximately 150 kDa, usually composed of two different kinds of polypeptide chains. One polypeptide chain, termed the heavy chain (H), is approximately 50 kDa. The other polypeptide, termed the light chain (L), is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each heavy and light chain also has regularly spaced intrachain disulfide bridges. Each light chain is linked to a heavy chain by one covalent disulfide bond. In any given naturally occurring antibody molecule, the two heavy chains have the same amino acid sequence, as do the two light chains, and each antibody harbors two identical antigen-binding sites, and are thus said to be divalent, i.e., having the capacity to simultaneously bind two identical molecules bearing the antigenic determinant targeted by the antibody. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibody molecules from any vertebrate species can be assigned to one of two clearly distinct types, kappa (k) and lambda (λ), based on the amino acid sequences of their constant domains. The ratio of the two types of light chain varies from species to species. As a way of example, the average k to λ ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes. The heavy-chain constant domains that correspond to the different classes of immunoglobulins are called alpha, delta, epsilon, gamma, and mu, respectively. The subunit structures and three-dimensional configurations of different classes of immunoglobulins are well known.

Of note, variability is not uniformly distributed throughout the variable domains of antibodies, but is concentrated in three segments called complementarity-determining regions (CDRs) or hypervariable regions, both in the light chain and the heavy chain variable domains. The more highly conserved portions of variable domains are called the framework regions (FR). The variable domains of native heavy and light chains each comprise four FR regions connected by three CDRs. The CDRs in each chain are held together in close proximity by the FR regions and, with the CDRs from the other chain, contribute to the formation of the antigen-binding site of antibodies (see Kabat, et al., above). Collectively, an antibody molecule's 6 CDRs contribute to its binding properties. However, even a single variable domain (or half of an Fv comprising only three CDRs specific for an antigen) has the ability to recognize and bind antigen (see Pluckthun, in The Pharmacology of Monoclonal Antibodies, vol. 113, Rosenburg and Moore, eds., Springer-Verlag, New York, pp. 269-315 (1994)).

The constant domain refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, “effector functions” refer to the different physiological effects of antibodies (e.g., opsonization, cell lysis, mast cell, basophil, and eosinophil degranulation, and other processes) mediated by the recruitment of immune cells by the molecular interaction between the Fc domain and proteins of the immune system. The isotype of the heavy chain determines the functional properties of the antibody. Antibodies' distinctive functional properties are conferred by the carboxy-terminal portions of the heavy chains, where they are not associated with light chains.

Antibody molecules can be tested for specificity of antigen binding by comparing binding to the desired antigen to binding to unrelated antigen or analogue antigen or antigen mixture under a given set of conditions. Preferably, an antibody will lack significant binding to unrelated antigens, or even analogs of the target antigen.

The term “antibody,” as used herein, is used in the broadest sense, and encompasses monoclonal, polyclonal, multispecific (e.g., bispecific, wherein each arm of the antibody is reactive with a different epitope of the same or different antigen), minibody, heteroconjugate, diabody, triabody, chimeric, and synthetic antibodies, as well as antibody fragments, derivatives, and variants that specifically bind an antigen with a desired binding property and/or biological activity.

Desired activities can include the ability to bind the target antigen specifically, the ability to inhibit proliferation in vitro, the ability to inhibit angiogenesis in vivo, the ability to alter cytokine profile(s) in vitro, the ability to modulate, particularly inhibit, receptor signaling mediated by the target antigen, etc. Other desired activities include increased stability, half-life, and/or bioavailability. In some embodiments, the antibody (or antigen-binding fragment thereof) is bound to polyethylene glycol (PEG).

### 4. Antibodies to LPA

The Examples below describe the production of anti-LPA antibodies with desirable properties from a therapeutic perspective, including: (a) binding affinity for LPA and/or its variants, including 18:2, 18:1, 18:0, 16:0, 14:0, 12:0, and 20:4 LPA. Antibody affinities may be determined as described in the Examples below or by any suitable now-known or later-developed method. Preferably, antibodies bind LPA with a high affinity, e.g., a K_{d} value of no more than about 1×10⁻⁷ M; preferably no more than about 1×10⁻⁸ M; and even more preferably, no more than about 5×10⁻⁹ M. In a physiological context, it is preferable for an antibody (or antigen-binding antibody fragment) to bind LPA with an affinity that is higher than LPA's affinity for one or more LPA receptors on cells in a subject to be treated. It will be understood that this need not necessarily be the case in a nonphysiological context such as a diagnostic assay. One assay format for determining the activity of the anti-LPA antibodies is ELISA.

Aside from antibodies (and antigen-binding antibody fragments) with strong binding affinity for LPA, it may also be desirable to select chimeric, humanized, or variant antibodies that have other beneficial properties from a therapeutic perspective. For example, the antibody may be one that reduces scar formation or increases neuronal differentiation. Preferably, the humanized or variant antibody (or antigen-binding antibody fragment) fails to elicit an immunogenic response upon administration of a therapeutically effective amount of the antibody to a human patient. If an immunogenic response is elicited, preferably the response will be such that the antibody still provides a therapeutic benefit to the patient treated therewith.

More information about antibodies to LPA, including antigen-binding antibody fragments and variants, can be found in commonly-owned patents and patent applications, e.g., U.S. Pat. No. 8,158,124 and U.S. patent application publication numbers 20080145360, 20100034814, and 20110076269, and in the Examples below. Antibodies (and antigen-binding antibody fragments) to LPA may be polyclonal or monoclonal, and may be humanized (or derived from humanized antibodies). Isolated nucleic acids encoding the heavy and light chains of an anti-LPA antibody, vectors and host cells comprising such nucleic acids, and recombinant techniques for the production of such antibodies, are also described in the above patents and patent applications. A number of nonlimiting examples of antibodies to LPA are shown in the Examples below.

### 5. Neuronal Differentiation and the Role of LPA

Neural stem cells (NSC) are found in areas of neurogenesis in the central nervous system (CNS) and can migrate to sites of neural injury. Thus, NSC are under study with the goal of replacing neurons and restoring connections in a neurodegenerative environment. Dottori, et al. (2008), Stem Cells 26: 1146-1154. NSC can be maintained in vitro as floating neurospheres and can differentiate in vitro into neurons. This can be assayed by visualizing and quantitating neuronal outgrowth from the neurospheres, which is visible under a microscope, or by any other suitable now-known or later-developed method.

Neuronal stem cells have the option of proceeding into neuronal differentiation or into glial differentiation (gliogenesis), the formation of non-neuronal glial cells. Macroglial cells (glia) include astrocytes and oligodendrocytes. Thus, in general, as neuronal differentiation increases, glial differentiation decreases and vice versa. Thus, an increase in neuronal differentiation may be determined by an increase in neuron formation, or by a decrease in glial differentiation.

Following injury, hemorrhage, or trauma to the nervous system, levels of LPA within the nervous system are believed to increase to 10 μM. Dottori, et al. (ibid) have shown that 10 μM LPA can inhibit neuronal differentiation of human NSC, while lower concentrations do not, suggesting that high levels of LPA within the CNS following injury might inhibit differentiation of NSC toward neurons, thus inhibiting endogenous neuronal regeneration. Modulating LPA signaling may thus have a significant impact in nervous system injury, allowing new potential therapeutic approaches. Antibodies to LPA are believed to decrease infarct, neuroinflammation (including gliogenesis), and neurodegeneration.

### 6. Applications

The instant application relates to methods for treating neurotrauma in a subject, as well as methods for decreasing infarct size in the brain following TBI and for increasing locomotor function recovery following TBI. These methods use antibodies or antibody fragments that bind to LPA. Thus, therapeutic uses of such molecules, particularly anti-LPA monoclonal antibodies, including the humanized antibody LT3114, are provided.

### 7. Formulations and Routes of Administration

Anti-LPA antibodies (and LPA-binding antibody fragments, variants, and derivatives) may be formulated in pharmaceutical compositions that are useful for a variety of purposes, including the treatment of neurotrauma, decreasing infarct size in the brain following TBI, and increasing locomotor function recovery following TBI. Pharmaceutical compositions comprising one or more anti-LPA antibodies (and/or LPA-binding antibody fragments) can be incorporated into kits and medical devices for such treatment. Medical devices may be used to administer the pharmaceutical compositions to a patient in need thereof, and according to one aspect, kits are envisioned that include such devices. Such devices and kits may be designed for routine administration, including self-administration, of such pharmaceutical compositions. Such devices and kits may also be designed for emergency use, for example, in ambulances or emergency rooms, or during surgery, or in activities where injury is possible but where full medical attention may not be immediately forthcoming (for example, hiking and camping, or combat situations).

Therapeutic formulations of an anti-LPA antibody (or LPA-binding antibody fragment) are prepared for storage by mixing the antibody (or antibody fragment) having the desired degree of purity with optional physiologically acceptable carriers, excipients, and/or stabilizers (see, e.g., Remington's Pharmaceutical Sciences 16th edition, Osol, A. Ed. (1980)), in the form of lyophilized formulations or aqueous solutions. Acceptable carriers, excipients, and/or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are described, for example, in Remington's Pharmaceutical Sciences 16th edition, Osol, A. Ed. (1980).

The formulations to be used for in vivo administration must be sterile. This is readily accomplished, for instance by filtration through sterile filtration membranes.

Sustained-release preparations can also be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved.

For therapeutic applications, the anti-LPA antibodies or antibody fragments are administered to a mammal, preferably a human, in a pharmaceutically acceptable dosage form such as those described above. Drug substances may be administered by techniques known in the art, including, but not limited to, systemic, subcutaneous, intradermal, mucosal, including by inhalation, and topical administration. Administration may be intravenous (either as a bolus or by continuous infusion over a period of time), or may be intramuscular, intraperitoneal, intra-cerebrospinal, epidural, intracerebral, intracerebroventricular, subcutaneous, intra-articular, intrasynovial, intrathecal, oral, topical, or by inhalation. Intranasal administration is also included, particularly via the rostral migratory stream (Scranton, et al. (2011), PLoS ONE 6:e18711. It has been shown that intranasal administration in mice allows agents to be distributed throughout the brain, circumventing the blood-brain barrier (Scranton, et al., ibid). Local administration (as opposed to systemic administration) may be advantageous because this approach can limit potential systemic side effects, but still allow therapeutic effect. One example of local administration is administration into the site of central nervous system (CNS) injury, such as into the site of a brain or spinal cord injury. For example, the biopolymer scaffold implant approach of Invivo Therapeutics allows drug release directly to the site of neurotrauma. George, et al. (2005), Biomaterials 26: 3511-3519.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg (microgram per kilogram) to about 50 mg/kg (e.g., 0.1-20 mg/kg (milligram per kg)) of antibody is an initial candidate dosage for administration to the patient, whether, for example, by one or more separate administrations, or by continuous infusion. A typical daily or weekly dosage might range from about 1 mg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. Detection methods using the antibody (or an LPA-binding antibody fragment) to determine LPA levels in bodily fluids or tissues may be used in order to optimize patient exposure to the therapeutic antibody.

According to another embodiment, the composition comprising an agent, e.g., a mAb that interferes with LPA activity, is administered as a monotherapy, while in other preferred embodiments, the composition comprising the agent that interferes with LPA activity is administered as part of a combination therapy. Preferred combination therapies include, in addition to administration of the composition comprising an agent that interferes with LPA activity, delivering a second therapeutic regimen such as administration of a second antibody or conventional drug, radiation therapy, surgery, and a combination of any of the foregoing. Such other agents may be present in the composition being administered or may be administered separately. Also, the antibody is suitably administered serially or in combination with the other agent or modality.

### EXAMPLES

The invention will be further described by reference to the following detailed examples. These Examples are in no way to be considered to limit the scope of the invention in any manner.

### Example 1

### Antibodies to LPA

Although polyclonal antibodies against naturally-occurring LPA have been reported in the literature (Chen, et al., Bioorg Med Chem Lett. 2000 Aug. 7:10(15)1691-3), monoclonal antibodies to LPA had not been described until Sabbadini, et al., U.S. patent application publication no. 20080145360, and U.S. Pat. No. 8,158,124, both of which are commonly owned herewith. The former publication describes the production and characterization of a series of murine monoclonal antibodies against LPA and the latter describes, among other things, a humanized monoclonal antibody against LPA. The specificity of each antibody for various LPA isoforms is shown in Table 1, below. IC₅₀: Half maximum inhibition concentration; MI: Maximum inhibition (% of binding in the absence of inhibitor); ---: not estimated because of weak inhibition. A high inhibition result indicates recognition of the competitor lipid by the antibody.

TABLE 1
Specificity profile of six anti-LPA mAbs [from U.S. Pub. No. 20080145360]

| | 14:0 LPA | | 16:0 LPA | | 18:1 LPA | | 18:2 LPA | | 20:4 LPA | |
|---|---|---|---|---|---|---|---|---|---|---|
| | IC₅₀ | MI | IC₅₀ | MI | IC₅₀ | MI | IC₅₀ | MI | IC₅₀ | MI |
| | uM | % | uM | % | uM | % | uM | % | uM | % |
| B3 | 0.02 | 72.3 | 0.05 | 70.3 | 0.287 | 83 | 0.064 | 72.5 | 0.02 | 67.1 |
| B7 | 0.105 | 61.3 | 0.483 | 62.9 | >2.0 | 100 | 1.487 | 100 | 0.161 | 67 |
| B58 | 0.26 | 63.9 | 5.698 | >100 | 1.5 | 79.3 | 1.240 | 92.6 | 0.304 | 79.8 |
| B104 | 0.32 | 23.1 | 1.557 | 26.5 | 28.648 | >100 | 1.591 | 36 | 0.32 | 20.1 |
| D22 | 0.164 | 34.9 | 0.543 | 31 | 1.489 | 47.7 | 0.331 | 31.4 | 0.164 | 29.5 |
| A63 | 1.147 | 31.9 | 5.994 | 45.7 | — | — | — | — | 0.119 | 14.5 |
| B3A6 | 0.108 | 59.9 | 1.151 | 81.1 | 1.897 | 87.6 | — | — | 0.131 | 44.9 |

Interestingly, the anti-LPA mAbs above were able to discriminate between 12:0 (lauroyl), 14:0 (myristoyl), 16:0 (palmitoyl), 18:1 (oleoyl), 18:2 (linoleoyl), and 20:4 (arachidonoyl) LPAs. A desirable EC₅₀ rank order for ultimate drug development is 18:2>18:1>20:4 for unsaturated lipids and 14:0>16:0>18:0 for the saturated lipids, along with high specificity. The specificity of the anti-LPA mAbs was assessed for their binding to LPA-related biolipids such as distearoyl-phosphatidic acid, lysophosphatidylcholine, S1P, ceramide, and ceramide-1-phosphate. None of the anti-LPA antibodies demonstrated cross-reactivity to distearoyl PA and LPC, the immediate metabolic precursor of LPA.

Tables 2-6, below, show primary amino acid sequences of the heavy and light chain variable domains (V_{H} and V_{L}) of five anti-LPA monoclonal antibodies.

TABLE 2
Clone B3 variable domain amino acid sequences without leader sequence and cut sites

| Sequence | SEQ ID NO: |
|---|---|
| B3 Heavy Chain | |
| QVKLQQSGPELVRPGTSVKVSCTASGDAFTNYLIEWVKQRPGQGLEWIGLIYPDSGYINYNENF | 1 |
| KGKATLTADRSSSTAYMQLSSLTSEDSAVYFCARRFAYYGSGYYFDYWGQGTTLTVSS | |
| B3 Light Chain | |
| DVVMTQTPLSLPVSLGDQASISCRSSQSLLKTNGNTYLHWYLQKPGQSPKLLIFKVSNRFSGVPD | 2 |
| RFSGSGSGTDFTLKISRVEAEDLGVYFCSQSTHFPFTFGTGTKLEIK | |

TABLE 3
Clone B7 variable domain amino acid sequences without leader sequence and cut sites

| Sequence | SEQ ID NO: |
|---|---|
| B7 Heavy Chain | |
| QVQLQQSGAELVRPGTSVKVSCKASGYGFINYLIEWIKQRPGQGLEWIGLINPGSDYTNYNENFK | 3 |
| GKATLTADKSSSTAYMHLSSLTSEDSAVYFCARRFGYYGSGNYFDYWGQGTTLTVSS | |
| B7 Light Chain | |
| DVVMTQTPLSLPVSLGDQASISCTSGQSLVHINGNTYLHWYLQKPGQSPKLLIYKVSNLFSGVPD | 4 |
| RFSGSGSGTDFTLKISRVEAEDLGVYFCSQSTHFPFTFGTGTKLEIK | |

TABLE 4
Clone B58 variable domain amino acid sequences without leader sequence and cut sites

| Sequence | SEQ ID NO: |
|---|---|
| B58 Heavy Chain | |
| QVQLQQSGAELVRPGTSVKVSCKASGDAFTNYLIEWVKQRPGQGLEWIGLIIPGTGYTNYNENF | 5 |
| KGKATLTADKSSSTAYMQLSSLTSEDSAVYFCARRFGYYGSSNYFDYWGQGTTLTVSS | |
| B58 Light Chain | |
| DVVMTQTPLSLPVSLGDQASISCRSSQSLVHSNGNTYLHWYLQKPGQSPKLLIYKVSNRFSGVP | 6 |
| DRFSGSGPGTDFTLKISRVEAEDLGIYFCSQSTHFPFTFGTGTKLEIK | |

TABLE 5
Clone 3A6 variable domain amino acid sequences without leader sequence and cut sites

| Sequence | SEQ ID NO: |
|---|---|
| 3A6 Heavy Chain | |
| QVQLQQSGAELVRPGTSVKLSCKASGDAFTNYLIEWVKQRPGQGLEWIGLIIPGTGYTNYNENF | 7 |
| KGKATLTADKSSSTAYMQLSSLTSEDSAVYFCARRFGYYGSGYYFDYWGQGTTLTVSS | |
| 3A6 Light Chain | |
| DVVMTQTPLSLPVSLGDQASISCRSSQSLVHSNGNTYLHWYLQKPGQSPKLLIYKVSNRFSGVP | 8 |
| DRFSGSGPGTDFTLKISRVEAEDLGVYFCSQSTHFPFTFGTGTKLEIK | |

TABLE 6
Clone A63 variable domain amino acid sequences without leader sequence and cut sites

| Sequence | SEQ ID NO: |
|---|---|
| A63 Heavy Chain | |
| DIQLQESGPGLVKPSQSLSLTCSVTGFSITSGYYWTWIRQFPGNKLEWVAYIGYDGSNDSNPSL | 9 |
| KNRISITRDTSKNQFFLKLNSVTTEDTATYYCARAMLRRGFDYWGQGTTLTVSS | |
| A63 Light Chain | |
| QIVLTQSPAIMSASPGEKVTMTCSASSSLSYMHWYQQKPGTSPKRWIYDTSKLASGVPARFSGS | 10 |
| GSGTSYSLTISSMEAEDAATYYCHRRSSYTFGGGTKLEIK | |

Tables 7-11, below, show the amino acid sequences of the CDRs of each of the antibodies represented in Tables 2-6, above.

TABLE 7
CDR amino acid sequences of V_{H} and V_{L} domains
for clone B3 of mouse anti-LPA mAb

| CLONE | | CDR | SEQ ID NO: |
|---|---|---|---|
| | V_{H} CDR | | |
| B3 | GDAFTNYLIE* | CDRH1 | 11 |
| B3 | LIYPDSGYINYNENFKG | CDRH2 | 12 |
| B3 | RFAYYGSGYYFDY | CDRH3 | 13 |
| | V_{L} CDR | | |
| B3 | RSSQSLLKTNGNTYLH | CDRL1 | 14 |
| B3 | KVSNRFS | CDRL2 | 15 |
| B3 | SQSTHFPFT | CDRL3 | 16 |

*CDRH1 as defined according to Chothia/AbM is the 10-amino acid sequence shown. The bolded five-amino acid portion (NYLIE; SEQ ID NO: 17) is the CDRH1 sequence defined according to Kabat.

TABLE 8
CDR amino acid sequences of V_{H} and V_{L} domains
for clone B7 of mouse anti-LPA mAb

| CLONE | | CDR | SEQ ID NO: |
|---|---|---|---|
| | V_{H} CDR | | |
| B7 | GYGFINYLIE* | CDRH1 | 18 |
| B7 | LINPGSDYTNYNENFKG | CDRH2 | 19 |
| B7 | RFGYYGSGNYFDY | CDRH3 | 20 |
| | V_{L} CDR | | |
| B7 | TSGQSLVHINGNTYLH | CDRL1 | 21 |
| B7 | KVSNLFS | CDRL2 | 22 |
| B7 | SQSTHFPFT | CDRL3 | 16 |

*CDRH1 as defined according to Chothia/AbM is the 10-amino acid sequence shown. The bolded five-amino acid portion (NYLIE; SEQ ID NO: 17) is the CDRH1 sequence defined according to Kabat.

TABLE 9
CDR amino acid sequences of V_{H} and V_{L} domains
for clone B58 of mouse anti-LPA mAb

| CLONE | | CDR | SEQ ID NO: |
|---|---|---|---|
| | V_{H} CDR | | |
| B58 | GDAFTNYLIE* | CDRH1 | 11 |
| B58 | LIIPGTGYTNYNENFKG | CDRH2 | 23 |
| B58 | RFGYYGSSNYFDY | CDRH3 | 24 |
| | V_{L} CDR | | |
| B58 | RSSQSLVHSNGNTYLH | CDRL1 | 25 |
| B58 | KVSNRFS | CDRL2 | 15 |
| B58 | SQSTHFPFT | CDRL3 | 16 |

*CDRH1 as defined according to Chothia/AbM is the 10-amino acid sequence shown. The bolded five-amino acid portion (NYLIE; SEQ ID NO: 17) is the CDRH1 sequence defined according to Kabat.

TABLE 10
CDR amino acid sequences of V_{H} and V_{L} domains
for clone 3A6 of mouse anti-LPA mAb

| CLONE | | CDR | SEQ ID NO: |
|---|---|---|---|
| | V_{H} CDR | | |
| 3A6 | GDAFTNYLIE* | CDRH1 | 11 |
| 3A6 | LIIPGTGYTNYNENFKG | CDRH2 | 23 |
| 3A6 | RFGYYGSGYYFDY | CDRH3 | 26 |
| | V_{L} CDR | | |
| 3A6 | RSSQSLVHSNGNTYLH | CDRL1 | 25 |
| 3A6 | KVSNRFS | CDRL2 | 15 |
| 3A6 | SQSTHFPFT | CDRL3 | 16 |

*CDRH1 as defined according to Chothia/AbM is the 10-amino acid sequence shown. The bolded five-amino acid portion (NYLIE; SEQ ID NO: 17) is the CDRH1 sequence defined according to Kabat.

TABLE 11
CDR amino acid sequences of V_{H} and V_{L} domains
for clone A63 of mouse anti-LPA mAb

| CLONE | | CDR | SEQ ID NO: |
|---|---|---|---|
| | V_{H} CDR | | |
| A63 | GFSITSGYYWT* | CDRH1 | 27 |
| A63 | YIGYDGSNDSNPSLKN | CDRH2 | 28 |
| A63 | AMLRRGFDY | CDRH3 | 29 |
| | V_{L} CDR | | |
| A63 | SASSSLSYMH | CDRL1 | 30 |
| A63 | DTSKLAS | CDRL2 | 31 |
| A63 | HRRSSYT | CDRL3 | 32 |

*CDRH1 as defined according to Chothia/AbM is the 11-amino acid sequence shown. The bolded six-amino acid portion (SGYYWT; SEQ ID NO: 33) is the CDRH1 sequence defined according to Kabat.

Biophysical Properties of Lpathomab/LT3000

Lpathomab/LT3000 (also referred to herein as the “B7” anti-LPA monoclonal antibody) has high affinity for the signaling lipid LPA (K_{D} of 1-50 pM as demonstrated by surface plasmon resonance in the BiaCore assay, and in a direct binding ELISA assay); in addition, LT3000 demonstrates high specificity for LPA, having shown no binding affinity for over 100 different bioactive lipids and proteins, including over 20 bioactive lipids, some of which are structurally similar to LPA. The murine antibody is a full-length IgG1k isotype antibody composed of two identical light chains and two identical heavy chains with a total molecular weight of 155.5 kDa. The biophysical properties are summarized in Table 12, below.

TABLE 12
General Properties of Monoclonal antibody B7,
also called Lpathomab or LT3000

| | |
|---|---|
| Identity | LT3000 (B7) |
| Antibody isotype | Murine IgG1k |
| Specificity | Lysophosphatidic acid (LPA) |
| Molecular weight | 155.5 kDa |
| OD of 1 mg/mL | 1.35 (solution at 280 nm) |
| K_{D} | 1-50 pM |
| Apparent Tm | 67° C. at pH 7.4 |
| Appearance | Clear if dissolved in 1x PBS buffer |
| | (6.6 mM phosphate, 154 mM sodium chloride, pH 7.4) |
| Solubility | >40 mg/mL in 6.6 mM phosphate, 154 mM |
| | sodium chloride, pH 7.4 |

Lpathomab has also shown biological activity in preliminary cell based assays such as cytokine release, migration and invasion; these are summarized below along with data showing specificity of LT3000 for LPA isoforms and other bioactive lipids, and in vitro biological effects of LT3000.

TABLE 13
Biologic properties of Monoclonal Antibody B7
LT3000 (Lpathomab, B7 antibody)

| | | 16:0 | 18:1 | | |
|---|---|---|---|---|---|
| A. Competitor Lipid | 14:0 LPA | LPA | LPA | 18:2 LPA | 20:4 LPA |
| IC₅₀ (mM) | 0.105 | 0.483 | >2.0 | 1.487 | 0.161 |
| MI (%) | 61.3 | 62.9 | 100 | 100 | 67 |

| B. Competitor Lipid | LPC | S1P | C1P | Cer | DSPA |
|---|---|---|---|---|---|
| MI (%) | 0 | 2.7 | 1.0 | 1 | 0 |

| | LPA | |
|---|---|---|
| C. Cell based assay | isoform | |
| | | % Inhibition |
| | | (over LPA taken as 100) |
| Migration | 18:1 | 35* |
| Invasion | 14:0 | 95* |
| IL-8 Release | 18:1 | 20 |
| IL-6 Release | 18:1 | 23* |
| | | % Induction |
| | | (over LPA + TAXOL taken as 100) |
| Apoptosis | 18:1 | 79 |

A. Competition ELISA assay was performed with Lpathomab and 5 LPA isoforms. 18:1 LPA was captured on ELISA plates. Each competitor lipid (up to 10 mM) was serially diluted in BSA/PBS and incubated with 3 nM Lpathomab. Mixtures were then transferred to LPA coated wells and the amount of bound antibody was measured.
B. Competition ELISA was performed to assess specificity of Lpathomab. Data were normalized to maximum signal (A₄₅₀) and were expressed as percent inhibition (n = 3). IC₅₀: half maximum inhibition concentration; MI %: maximum inhibition (% of binding in the absence of inhibitor).
C. Migration assay: Lpathomab (150 mg/mL) reduced SKOV3 cell migration triggered by 1 mM LPA (n = 3); Invasion assay: Lpathomab (15 mg/mL) blocked SKOV3 cell invasion triggered by 2 mM LPA (n = 2); Cytokine release of human IL-8 and IL-6: Lpathomab (300-600 mg/mL, respectively) reduced 1 mM LPA-induced release of pro-angiogenic and metastatic IL-8 and IL-6 in SKOV3 conditioned media (n = 3). Apoptosis: SKOV3 cells were treated with 1 mM Taxol; 1 mM LPA blocked Taxol induced caspase-3 activation. The addition to Lpathomab (150 mg/mL) blocked LPA-induced protection from apoptosis (n = 1).
Data Analysis: Student-t test,
*denotes p < 0.05.

The potent and specific binding of Lpathomab/LT3000 to LPA results in reduced availability of extracellular LPA with potentially therapeutic effects against cancer-, angiogenic- and fibrotic-related disorders.

A second murine anti-LPA antibody, B3, was also subjected to binding analysis as shown in Table 14, below.

TABLE 14
Biochemical characteristics of Monoclonal Antibody B3
Biochemical characteristics of B3 antibody

| A. BIACORE | High density surface | Low density surface |
|---|---|---|
| Lipid Chip | 12:0 LPA | 18:0 LPA |
| K_{D} (pM), site 1 (site2) | 61(32) | 1.6 (0.3) |

| B. Competition Lipid Cocktail | |
|---|---|
| (C₁₆:C₁₈:C_{18:1}:C_{18:2}:C_{20:4,} | |
| ratio 3:2:5:11:2) | (μM) |
| IC₅₀ | 0.263 |

| C. Neutralization Assay | |
|---|---|
| B3 antibody (nmol) | LPA (nmol) |
| 0 | 0.16 |
| 0.5 | 0.0428 |
| 1 | 0.0148 |
| 2 | under limit of detection |

A. Biacore analysis for B3 antibody. 12:0 and 18:0 isoforms of LPA were immobilized onto GLC sensor chips; solutions of B3 were passed over the chips and sensograms were obtained for both 12:0 and 18:0 LPA chips. Resulted sensograms showed complex binding kinetics of the antibody due to monovalent and bivalent antibody binding capacities. K_{D} values were calculated approximately for both LPA 12 and LPA 18.
B. Competition ELISA assay was performed with B3 and a cocktail of LPA isoforms (C₁₆:C₁₈:C_{18:1}:C_{18:2}:C_{20:4} in ratio 3:2:5:11:2). Competitor/Cocktail lipid (up to 10 μM) was serially diluted in BSA/PBS and incubated with 0.5 μg/mL B3. Mixtures were then transferred to a LPA coated well plate and the amount of bound antibody was measured. Data were normalized to maximum signal (A₄₅₀) and were expressed as IC₅₀ (half maximum inhibition concentration).
C. Neutralization assay: Increasing concentrations of B3 were conjugated to a gel. Mouse plasma was then activated to increase endogenous levels of LPA. Activated plasma samples were then incubated with the increasing concentrations of the antibody-gel complex. LPA leftover that did not complex to the antibody was then determined by ELISA. LPA was sponged up by B3 in an antibody concentration dependent way.

### Humanization of LT3000

The variable domains of the B7 murine anti-LPA monoclonal antibody (LT3000, Lpathomab) were humanized by grafting the murine CDRs into human framework regions (FR). See, e.g., commonly-owned U.S. Pat. No. 8,604,172. For descriptions of CDR grafting techniques, see, for example, Lefranc, M. P (2003), Nucleic Acids Res, 31: 307-10; Martin and Thornton (1996), J Mol Biol, 1996. 263: 800-15; Morea, et al. (2000), Methods, 20: 267-79; Foote and Winter (1992), J Mol Biol, 224: 487-99; Chothia, et al., (1985). J Mol Biol, 186:651-63.

Suitable acceptor human FR sequences were selected from the IMGT and Kabat databases based on a homology to LT3000 using a sequence alignment and analysis program (SR v7.6). Lefranc (2003), supra; Kabat, et al. (1991), above, pp. 1-3242. Sequences with high identity at FR, vernier, canonical and V_{H}-V_{L} interface residues (VCI) were initially selected. From this subset, sequences with the most non-conservative VCI substitutions, unusual proline or cysteine residues, and somatic mutations were excluded. AJ002773 was thus selected as the human framework on which to base the humanized version of LT3000 heavy chain variable domain and DQ187679 was thus selected as the human framework on which to base the humanized version of LT3000 light chain variable domain.

A three-dimensional (3D) model containing the humanized V_{H} and V_{L} sequences was constructed to identify FR residues juxtaposed to residues that form the CDRs. These FR residues potentially influence the CDR loop structure and the ability of the antibody to retain high affinity and specificity for the antigen. Based on this analysis, six residues in AJ002773 and three residues in DQ187679 were identified, deemed significantly different from LT3000, and considered for mutation back to the murine sequence.

The sequence of the murine anti-LPA mAb LT3000 was humanized with the goal of producing an antibody that retained high affinity, specificity, and binding capacity for LPA. Further, seven humanized variants were transiently expressed in HEK 293 cells in serum-free conditions, purified, and then characterized in a panel of assays. Plasmids containing sequences of each light chain and heavy chain were transfected into mammalian cells for production. After 5 days of culture, mAb titers were determined using quantitative ELISA. All combinations of the heavy and light chains yielded between 2-12 ug of antibody per ml of cell culture.

Characterization and Activity of the Humanized Variants

All the humanized anti-LPA mAb variants exhibited binding affinity in the low picomolar range similar to a chimeric anti-LPA antibody (also known as LT3010) and the murine antibody LT3000. All of the humanized variants exhibited a T_{M} similar to or higher than that of LT3000. With regard to specificity, the humanized variants demonstrated similar specificity profiles to that of LT3000. For example, LT3000 demonstrated no cross-reactivity to lysophosphatidyl choline (LPC), phosphatidic acid (PA), various isoforms of lysophosphatidic acid (14:0 and 18:1 LPA, cyclic phosphatidic acid (cPA), and phosphatidylcholine (PC).

Five humanized variants were further assessed in in vitro cell assays. LPA is important in eliciting release of interleukin-8 (IL-8) from cancer cells. LT3000 reduced IL-8 release from ovarian cancer cells in a concentration-dependent manner. The humanized variants exhibited a similar reduction of IL-8 release compared to LT3000.

Two humanized variants were also tested for their effect on microvessel density (MVD) in a Matrigel tube formation assay for neovascularization. Both were shown to decrease MVD formation.

Humanized Anti-LPA Variable Region Sequences

The humanized variant sequences are shown in Tables 15 and 17, below. Backmutations are shown in bold. CDR sequences are shown in gray. Canonical residues are numbered according to which CDR (1, 2, or 3) with which they are associated. Additional sequence information is provided in commonly-owned U.S. patent application publication no. 20110076267.

TABLE 15
Sequences of the variable domains of anti-LPA light chain humanized variants.
CDRs are shaded; backmutations are in bold.

| | | SEQ |
|---|---|---|
| | | ID |
| | VK sequence | NO: |
| Canon- | 1 1 1 1 1 2 | N/A |
| ical | 2 1 3 3 3 | |
| Vernier | * * * * **** | N/A |
| | * * ** * * | |
| Inter- | F F F F F | N/A |
| face | F F F F | |
| Kabat number | 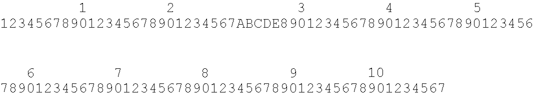 | N/A |
| B7 VK murine | 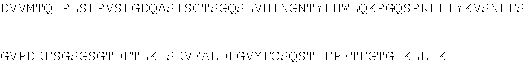 | 4 |
| B7RKA | 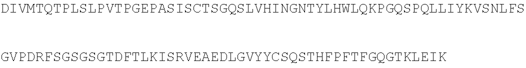 | 34 |
| B7RKB | 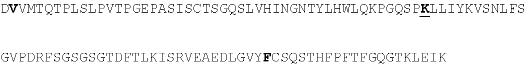 | 35 |
| B7RKC | 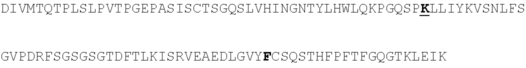 | 36 |
| B7RKD | 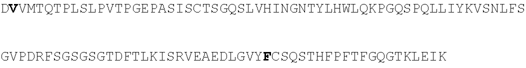 | 37 |
| B7RKE | 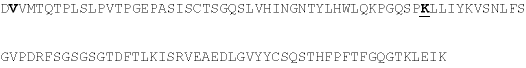 | 38 |
| B7RKF | 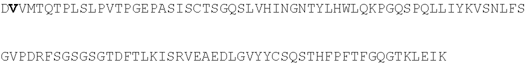 | 39 |
| B3-700 | 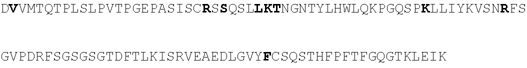 | 40 |
| B3-701 | 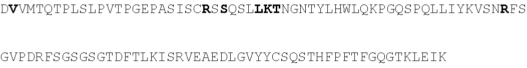 | 41 |
| B3-702 | 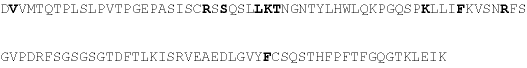 | 42 |

TABLE 16
LPA humanized antibody light chain variant variable domain sequences and vectors containing them.

| | | Number of | |
|---|---|---|---|
| Vector name | Description | backmutations | Identity of backmutations |
| pATH500LC | pCONkappa (Lonza vector alone) | N/A | N/A |
| pATH501 | B7 humanized light chain RKA in vector pATH500LC, no back mutations | 0 | N/A |
| pATH502 | B7 humanized light chain RKB in vector pATH500, 3 back mutations | 3 | I2V, Q45K, Y87F |
| pATH503 | B7 humanized light chain RKC in vector pATH500, 2 back mutations | 2 | Q45K, Y87F |
| pATH504 | B7 humanized light chain RKD in vector pATH500, 2 back mutations | 2 | I2V, Y87F |
| pATH505 | B7 humanized light chain RKE in vector pATH500, 2 back mutations | 2 | I2V, Q45K |
| pATH506 | B7 humanized light chain RKF in vector pATH500, 1 back mutation | 1 | I2V |
| pATH700 | B3 humanized light chain B3-700 in vector pATH500 | 9 | I2V, T24R, G26S, V27cL, H27dK, I27eT, |
| | | | Q45K, L54R, Y87F |
| pATH701 | B3 humanized light chain B3-701 in vector pATH500 | 7 | I2V, T24R, G26S, V27cL, H27dK, I27eT, |
| | | | L54R, |
| pATH702 | B3 humanized light chain B3-702 in vector pATH500 | 10 | I2V, T24R, G26S, V27cL, H27dK, I27eT, |
| | | | Q45K, Y49F, L54R, Y87F |

TABLE 17
Sequences of the variable domains of anti-LPA heavy chain humanized variants.
CDRs are shaded; backmutations are in bold

| | | SEQ |
|---|---|---|
| | | ID |
| Kabat # | 1 | NO: |
| | 1234567890123456789012345678901234567890123456789012A34567890123456 | N/A |
| | 7890123456789012ABC345678901234567890ABCDK1234567890123 | |
| Canonical | 1 11 1 1 2 22 | N/A |
| | 2 1 | |
| Vernier | * **** *** | N/A |
| | * * * * * ** * | |
| Interface | I I I I I | N/A |
| | I I I I I | |
| B7 VH murine | 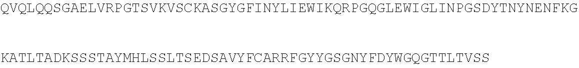 | 3 |
| B7RH0 | 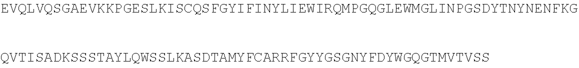 | 43 |
| B7RH1 | 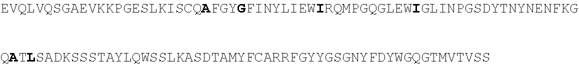 | 44 |
| B7RH2 | 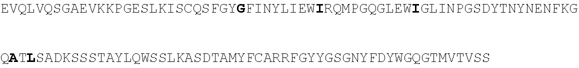 | 45 |
| B7RH3 | 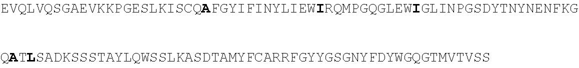 | 46 |
| 47B7RH4 | 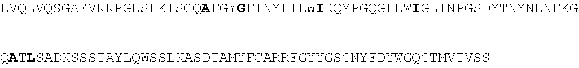 | 47 |
| B7RH5 | 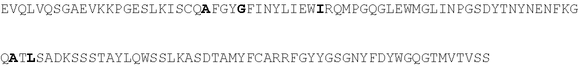 | 48 |
| B7RH6 | 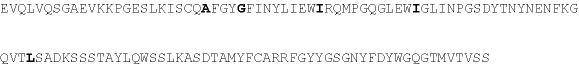 | 49 |
| B7RH7 | 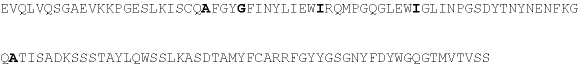 | 50 |
| B7RH8 | 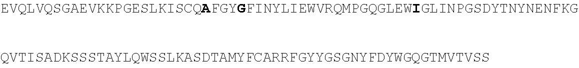 | 51 |
| B7RH9 | 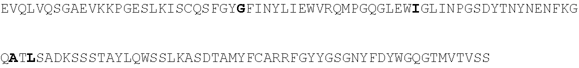 | 52 |
| B7HX | 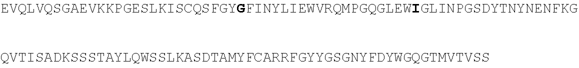 | 53 |
| B7HY | 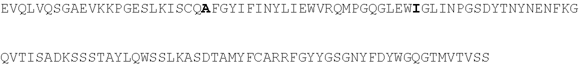 | 54 |
| B6HZ | 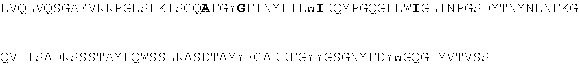 | 55 |
| B7-608 | 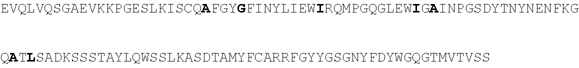 | 56 |
| B3-800 | 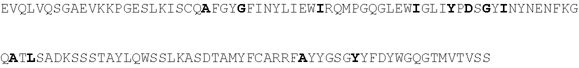 | 57 |
| B3-801 | 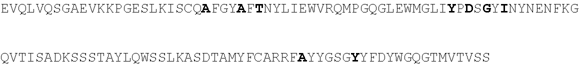 | 58 |
| B3-802 | 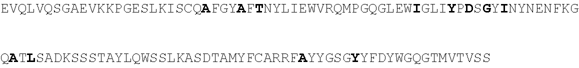 | 59 |
| B3-803 | 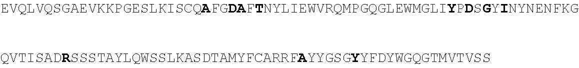 | 60 |
| B3-804 | 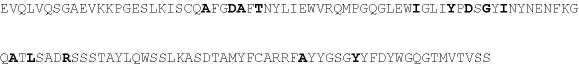 | 61 |

TABLE 18
LPA humanized antibody heavy chain variant variable domain sequences and vectors containing them.

| | | Number of | |
|---|---|---|---|
| Vector name | Description | backmutations | Identity of backmutations |
| pATH600HC | pCONgamma (Lonza vector alone) | N/A | N/A |
| pATH601 | B7 humanized heavy chain RH0 in vector pATH600 | 0 | N/A |
| pATH602 | B7 humanized heavy chain RH1 in vector pATH600 | 6 | S24A, I28G, V37I, M48I, V67A, I69L |
| pATH603 | B7 humanized heavy chain RH8 in vector pATH600 | 3 | S24A, I28G, M48I |
| pATH604 | B7 humanized heavy chain RH9 in vector pATH600 | 4 | I28G, M48I, V67A, I69L |
| pATH605 | B7 humanized heavy chain HX in vector pATH600 | 2 | I28G and M48I |
| pATH606 | B7 humanized heavy chain HY in vector pATH600 | 2 | S24A and M48I |
| pATH607 | B7 humanized heavy chain HZ in vector pATH600 | 4 | S24A, I28G, V37I, M48I |
| pATH608 | B7 humanized heavy chain B7-608 in vector pATH600 | 7 | S24A, I28G, V37I, M48I, L50A, V67A, |
| | | | I69L, |
| pATH800 | B3 humanized heavy chain B3-800 in vector pATH600 | 12 | S24A, I28G, V37I, M48I, N52Y, G53D, |
| | | | D55G, T57I, V67A, I69L, G97A, N100cY |
| pATH801 | B3 humanized heavy chain B3-801 in vector pATH600 | 9 | S24A, I28A, I30T, N52Y, G53D, D55G, |
| | | | T57I, G97A, N100cY |
| pATH802 | B3 humanized heavy chain B3-802 in vector pATH600 | 12 | S24A, I28A, I30T, M48I, N52Y, G53D, |
| | | | D55G, T57I, V67A, I69L, G97A, N100cY |
| pATH803 | B3 humanized heavy chain B3-803 in vector pATH600 | 11 | S24A, Y27D, I28A, I30T, N52Y, G53D, |
| | | | D55G, T57I, K73R, G97A, N100cY |
| pATH804 | B3 humanized heavy chain B3-804 in vector pATH600 | 14 | S24A, Y27D, I28A, I30T, M48I, N52Y, |
| | | | G53D, D55G, T57I, V67A, I69L, K73R, |
| | | | G97A, N100cY |

LT3015

LT3015 is a recombinant, humanized, monoclonal antibody that binds with high affinity to the bioactive lipid lysophosphatidic acid (LPA). LT3015 is a full-length IgG1k isotype antibody composed of two identical light chains and two identical heavy chains with a total molecular weight of 150 kDa. The heavy chain contains an N-linked glycosylation site. The two heavy chains are covalently coupled to each other through two intermolecular disulfide bonds, consistent with the structure of a human IgG1.

LT3015 was originally derived from a murine monoclonal antibody that was produced using hybridomas generated from mice immunized with LPA. The humanization of the murine antibody involved the insertion of the six murine complementarity-determining regions (CDRs) in place of those of a human antibody framework selected for its structure similarity to the murine parent antibody. A series of substitutions were made in the framework to engineer the humanized antibody. These substitutions are called “back mutations” and replace amino acid residues at the particular amino acid positions in the human antibody with corresponding murine residues that are involved in the interaction with the antigen. The final humanized version, designated “LT3015,” contains six murine back mutations in the human heavy chain variable domain framework (encoded by pATH602) and three murine back mutations in the human light chain variable domain framework (encoded by pATH502), as shown in Tables 15-18, above.

The variable domains of the humanized anti-LPA monoclonal antibody were then cloned into the vector IgG1k of the Lonza Biologics' GS gene expression system to generate the vector pATH3015. This expression system consists of an expression vector carrying the constant domains of the heavy and light chain genes and the selectable marker glutamine synthetase (GS; an enzyme that catalyzes the biosynthesis of glutamine from glutamate and ammonia). The vector carrying both the heavy and light genes and the selectable marker were transfected into CHOK1SV, a Chinese Hamster Ovary cell line providing sufficient glutamine for cells to survive without exogenous glutamine. In addition, the specific GS inhibitor, methionine sulphoximine (MSX), was supplemented in the medium to inhibit endogenous GS activity such that only the cell lines with GS activity provided by the vector could survive. The transfected cells were selected for their ability to grow in glutamine-free medium in the presence of MSX.

pATH3016 was produced similarly to pATH3015. As described above, the heavy chains of pATH3015 and 3016 are identical (derived from pATH602, having six backmutations), but the pATH3016 light chain (derived from pATH506) contains only a single backmutation, I2V. The humanized monoclonal antibody produced from pATH3016 was designated “LT3016”. Both pATH3015 and pATH3016 were deposited with the American Type Culture Collection (Manassas Va.) and have ATCC Patent Deposit Designations PTA-9219 and PTA-9220, respectively.

LT3114 is another recombinant, humanized, monoclonal antibody that binds LPA with high affinity. In contrast to LT3015 and LT3016, LT3114 was originally derived by humanization of the murine monoclonal antibody B3, using methods described above. In LT3144, the heavy chain variable domain has the amino acid sequence of SEQ ID NO: 61 (shown in Table 17, above, as B3-804 and further described in Table 18, above) and the light chain variable domain amino acid sequence of SEQ ID NO: 42 (shown in Table 15, above, as B3-702 and further described in Table 16, above).

### Example 2

### Neurosphere Formation, Differentiation, and Modeling

Neurospheres were used to model the role of LPA in neuronal differentiation as described in commonly-owned U.S. patent application publication no. US20110076269 and U.S. Pat. No. 8,604,172. Neurospheres were formed and cultured as described in Dottori, et al. (2008), supra, which also shows that LPA inhibits the ability of NSC to form neurospheres, even in the presence of bFGF and EGF. LPA also interferes with an additional differentiation step, namely, the differentiation of NSC toward mature cells.

Anti-LPA antibodies have been found by the inventors to block LPA inhibition of neurosphere formation, as described in commonly-owned U.S. patent application publication no. US20110076269 and U.S. Pat. No. 8,604,172. Noggin-treated cells incubated with the anti-LPA antibody B3 alone gave neurosphere formation comparable to control, and, notably, the combination of 1 mg/ml B3 and 10 μM LPA also gave neurosphere formation comparable to control, indicating that the antibody to LPA had blocked inhibition of neurosphere formation that normally occurs in the presence of LPA. Cells treated with the combination of LPA and the humanized anti-LPA antibody LT3015 showed nearly identical neuron formation to B3-treated cells.

LPA also inhibits the neuronal differentiation of adult mouse NSC, although, contrary to what was observed in human NSC, LPA did not modify neurosphere formation or growth of mouse NSC (for details, see U.S. patent application publication no. US20110076269 and U.S. Pat. No. 8,604,172).

Adult NS/PC can be used to elucidate LPA's effect in neurotrauma. Adult NS/PC are present in the central nervous system, predominantly in neurogenic regions such as the subventricular zone (SVZ) and hippocampus. They have been reported to migrate to sites of injury and tumors, effects likely to be linked to the repair of damaged tissue. Furthermore, it was recently shown that NS/PC contribute to neurogenesis in the adult mouse following stroke. Jin, et al. (2010), Proc Natl Acad Sci USA 107:7993-8. In vivo, it is expected that when LPA levels increase following trauma, such elevation would limit neuronal regeneration in the CNS. Thus, the inventors believe that antibodies that neutralize LPA will be useful in promoting neurogenesis following CNS injury.

The progressive differentiation of human embryonic stem cells (hESC) toward neural derivatives (i.e., NS/PC, neurons, and glia) makes possible the assessment of their responses to treatments of interest, thereby allowing the in vitro modelling of specific physiopathological events, particularly inflammation and trauma. Such in vitro modelling of neurotrauma using human stem cells and derivatives has allowed the study of not only NS/PC but also neurons and glial cells and how they respond to LPA and, in particular, to the high concentrations of LPA observed during neurotrauma (Dottori and Pera (2008), Methods Mol Biol 438:19-30; Dottori, et al. (Stem Cells (2008), 26:1146-54; U.S. patent application publication nos. US20110076269 and US20120128666). Considering the pleiotropic effects of LPA on most neural cell types, including NS/PC, together with data showing localized upregulation of LPARs following injury in both mice and humans, it is believed that LPA regulates essential aspects of cellular reorganization following neural trauma through its effects on reactive astrogliosis (glial response) and/or glial scarring), neural degeneration, and NS/PC migration and differentiation. Thus, the inventors believe that LPA is a key player in regulating response to injury and thus in modulating the outcome of CNS damage.

### Example 3

### Humanized and Murine Anti-LPA Antibodies Block LPA Inhibition of Neuronal Differentiation

Using the same conditions used in Example 2, above, for LPA treatment alone, plated neurospheres were treated with 10 μM LPA alone, or with anti-LPA antibody B3 or B7 (1 mg/ml) alone, or with 10 μM LPA in combination with 1 mg/ml of antibody B3 or B7. Similarly, cells were treated with 10 μM LPA alone, humanized anti-LPA antibody LT3015 alone (1 mg/ml), or with 10 μM LPA in combination with 1 mg/ml LT3015. The percent of neuron-forming neurospheres was quantitated as in Example 2 (beta-tubulin staining and quantification of neuron-forming spheres, as described in Dottori, et al (2008)). LPA alone reduced neuron-forming neurospheres to approximately 25.00±6.45% of untreated control. Neurosphere samples treated with B3 antibody alone had neuron-forming neurospheres equivalent to control (100%). Neurospheres treated with the combination of LPA and B3 antibody had neuron forming neurospheres equal to 86.66±5.65% of control, indicating that the antibody had blocked the inhibition of neuron formation that normally occurs in the presence of LPA. Cells treated with the combination of LPA and LT3015 humanized antibody showed nearly identical neuron formation to B3-treated cells (87.5%±12.50% of control). The antibody B7, under similar conditions, had little to no effect in this experiment (37.00±5.31% of control).

### Example 4

### Humanized and Murine Anti-LPA Antibodies Block LPA Inhibition of Neurosphere Formation

Using the conditions described in the Example above, HSC were plated onto laminin for neuronal differentiation in NBM medium (3 days), with or without LPA (10 μM), with or without antibody to LPA at 1 mg/ml (B3, B7, or the humanized antibody LT3015, tested singly with or without LPA).

As before, the number of neuron-forming spheres was significantly decreased in the presence of 10 μM LPA, to approximately 26% of control. None of the antibodies when tested alone had any effect on number of neuron-forming spheres (all were equivalent to control, which was 100%). However, all of the anti-LPA antibodies were able to block the inhibition of neuronal differentiation by LPA. Cells treated with B3 and LPA or with LT3015 and LPA had neuron-forming neurospheres equal to 75% of control. Cells treated with B7 and LPA had neuron-forming neurospheres equal to 50% of control. Pool of data results are similar: LPA 25.00±6.45%; B3+LPA: 86.66±5.65; B7+LPA 37.00±5.31%; humanized B7 (LT3015): 87.5±12.5 (however, although differentiation was observed, there were fewer neurons observed than with B3) n=2 for hB7 and n>3 for B3 and B7. Thus, all three LPA antibodies, including LT3015, inhibited LPA's effect on neuronal differentiation, as measured by neurosphere formation. It was noted that neurospheres from cells treated with B3 and LPA had the greatest number of neurons (indicating further differentiation), followed by neurospheres from LT3015-treated cells, with a lesser number of neurons in neurospheres from cells treated with B7 antibody.

### Example 5

### Use of NS/PC for the Understanding of LPA's Effect in Neurotrauma

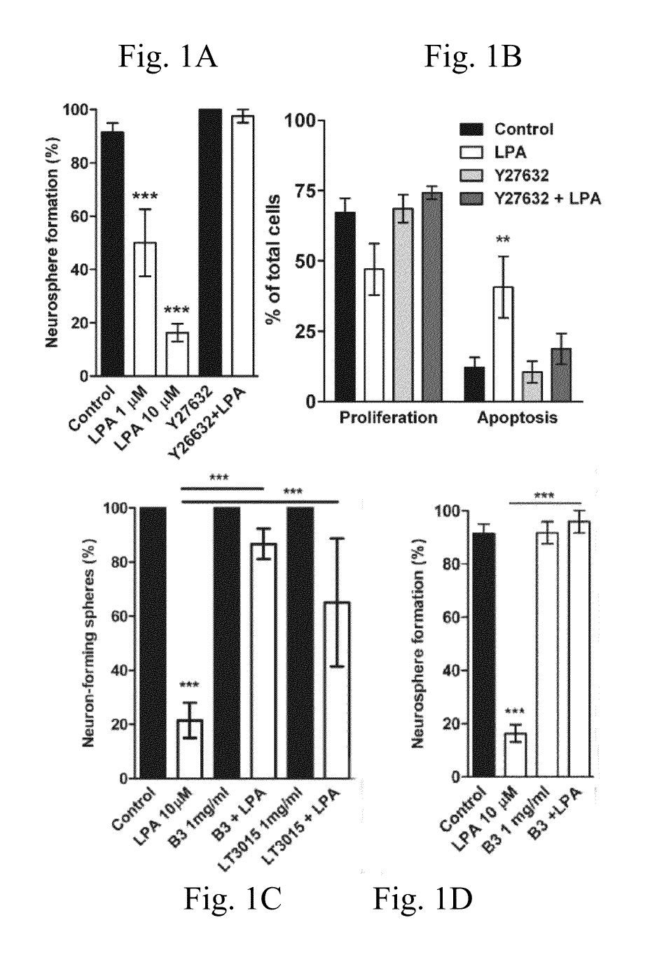

Adult NS/PC are present in the central nervous system, predominantly in neurogenic regions such as the subventricular zone (SVZ) and hippocampus. They have been reported to migrate to sites of injury and tumors, effects likely to be linked to the repair of damaged tissue. Furthermore, it was recently shown that NS/PC contribute to neurogenesis in the adult mouse following stroke. Jin, et al., supra. LPA inhibits the neuronal differentiation of mouse adult NS/PC (mNS/PC) of SVZ origin, as shown in FIG. 1. In vivo, it is expected that when LPA levels increase following trauma, such elevation would limit neuronal regeneration in the CNS. Thus, antibodies that neutralize LPA are believed to be useful in promoting neurogenesis following CNS injury.

Aside from their presence in the CNS, NS/PC can also be used for in vitro modelling. Indeed, the progressive differentiation of human embryonic stem cells (hESC) towards their neural derivatives (i.e., NS/PC, neurons, and glia) makes possible the assessment of their responses to treatments of interest; hence it allows the in vitro modelling of specific physiopathological events, particularly inflammation and trauma. This in vitro modelling of neurotrauma using human stem cells and derivatives has allowed the study of not only NS/PC but also neurons and glial cells (all of which can be studied together in our differentiation assays) and how they respond to LPA and, in particular, to the high concentrations of LPA observed during neurotrauma (Dottori and Pera (2008), Methods Mol Biol 438:19-30). Dottori, et al. (Stem Cells (2008), 26:1146-54) demonstrated that LPA specifically inhibits the differentiation of NS/PC towards neurons while maintaining their differentiation toward astrocytes, and that LPA's effect on NS/PC can be abolished by specific anti-LPA mAbs (B3, LT3015). As shown in FIG. 1, addition of 10 uM LPA to neurospheres resulted in a nearly 80% decrease in neuron-forming spheres. This effect was completely blocked by addition of the murine anti-LPA antibody B3 or the humanized anti-LPA antibody LT3015 (1 mg/ml) for three days. n≧3 independent experiments. Neurosphere formation was also inhibited by LPA (10 uM), and this effect was entirely abolished by addition of the murine B3 anti-LPA antibody at 1 mg/ml, even if added in combination with LPA.

These data indicate that high levels of LPA within the CNS following an injury inhibit endogenous neurogenesis by inducing NS/PC apoptosis, by blocking their neuronal differentiation and by promoting gliosis. These data also highlight the potency of anti-LPA mAbs in blocking LPA. Considering the pleiotropic effects of LPA on most neural cell types, including NS/PC, together with data showing localized upregulation of LPA receptors following injury in both mice and humans, it is believed that LPA regulates essential aspects of cellular reorganization following neural trauma through its effects on reactive astrogliosis (glial response) and/or glial scarring, neural degeneration, and NS/PC migration and differentiation. Thus, the inventors believe that LPA is a key component in regulating response to injury and thus in modulating the outcome of CNS damage.

### Example 6

### Immunohistochemical Staining of LPA Using Monoclonal Anti-LPA Antibodies

Immunohistochemical methods can be used to determine the presence and location of LPA in cells. Spinal cords from animals (adult (3 mo. Old, 20-30 g) male C57BL/6 mice) with and without spinal cord injury (SCI) were immunostained 4 days after injury. Mice were anaesthetized with a mixture of ketamine and xylazine (100 mg/kg and 16 mg/kg, respectively) in phosphate buffered saline (PBS) injected intraperitoneally. The spinal cord was exposed at the low thoracic to high lumbar area, at level T12, corresponding to the level of the lumbar enlargement. Fine forceps were used to remove the spinous process and lamina of the vertebrae and a left hemisection was made at T12. A fine scalpel was used to cut the spinal cord, which was cut a second time to ensure that the lesion was complete, on the left side of the spinal cord, and the overlying muscle and skin were then sutured. This resulted in paralysis of the left hindlimb. After 2 or 4 days the animals were re-anaesthetized as above and then perfused with PBS through the left ventricle of the heart, followed by 4% paraformaldehyde (PFA). After perfusion, the spinal cords were gently removed using fine forceps and post-fixed for 1 hour in cold 4% PFA followed by paraffin embedding or cryo-preserving in 20% sucrose in PBS overnight at 4° C. for frozen sections. Tissues for taken from n=3 uninjured mice and n=3 injured mice at 2 and 4 days post-injury. See Goldshmit, et al. (2004), J Neurosci 2004, 24(45):10064-10073.

IHC frozen spinal cord sagittal sections (10 μm) were examined using standard immunohistochemical procedures to determine expression and localization of the different LPA receptors. Frozen sections were postfixed for 10 min. with 4% PFA and washed 3 times with PBS before blocking for 1 hour at room temperature (RT) in blocking solution containing 5% goat serum (Millipore) and 0.1% Triton-X in PBS in order to block non-specific antisera interactions. Primary antibodies used were B3 (0.1 mg/ml), rabbit anti-LPA₁ (1:100, Cayman Chemical, USA), rabbit anti-LPA₂ (1:100, Abcam, UK) and mouse anti-GFAP (1:500, Dako, Denmark). Primary antibodies were added in blocking solution and sections incubated over night at 4° C. The sections were then washed and incubated in secondary antibody for 1 hour at room temperature, followed by Dapi counterstaining. Sections were coverslipped in Fluoromount (Dako) and examined using an Olympus BX60 microscope with a Zeiss Axiocam HRc digital camera and Zeiss Axiovision 3.1 software to capture digital images. Some double-labeled sections were also examined using a Biorad MRC1024 confocal scanning laser system installed on a Zeiss Axioplan 2 microscope. All images were collated and multi-colored panels were produced using Adobe Photoshop 6.0.

After injury, astrocytes (non-neuronal glial cells in the CNS) respond to many damage and disease states resulting in a “glial response”. Glial Fibrillary Acidic Protein (GFAP) antibodies are widely used to detect reactive astrocytes that form part of this response, since reactive astrocytes stain much more strongly with GFAP antibodies than do normal astrocytes. LPA was revealed by immunohistochemistry using antibody B3 (0.1 mg/ml overnight). Fluorescence microscopy showed that reactive astrocytes were present in spinal cords 4 days after injury, and these cells stained positively for LPA. In contrast, uninjured (control) spinal cords had little to no staining for astrocytes or LPA. Thus, LPA was present in reactive astrocytes of the spinal cord. In both injured and control animals, the central canal (a potential stem cell niche) did not stain for LPA.

### Example 7

### Immunohistochemical Confirmation that Anti-LPA Antibodies Block LPA Inhibition of Neuronal Differentiation

Neurospheres grown and treated as in the Examples above were immunostained for CD133 (1/1000, Abcam, Inc., Cambridge Mass.), β-tubulin (1/500, Millipore, Billerica Mass.), or LPA (0.1 mg/ml) as described in the Example 6, above. β-tubulin staining is indicative of differentiation of neurons. In contrast, CD133 staining is lost upon differentiation. With LPA treatment, CD133-positive cells are observed as the cells migrating out of the neurosphere. In control cells, the migrating cells were either weakly CD133 positive or were negative for CD133 staining. Expression of CD133 was qualitatively observed to be reduced by the LPA antibodies.

### Example 8

### LPA Inhibits the Neuronal Differentiation of Adult Mouse NSC

In mouse adult neurospheres generated from mouse subventricular zone NSC, expression analysis of the LPA receptors indicated the presence of the mRNA transcripts for LPA receptors LPA₁, LPA₃, and LPA₄ and absence or low level expression of mRNA transcripts for the LPA receptors LPA₂ and LPA₅, indicating that adult mNS/PC are also potential targets for LPA. Contrary to what was observed in human NSC, LPA did not modify neurosphere formation or growth of mouse NSC. However, and similarly to data obtained in human NSC, LPA inhibited the neuronal differentiation of adult mouse NSC by maintaining them as NSC when plated in conditions normally inducing neuronal differentiation. After three days, LPA (10 μM) -treated mouse NSC only showed low levels of expression of βIII-tubulin, a marker for differentiated neurons (26.25±2.08% of total cells), and remained mainly positive for nestin, a marker for undifferentiated NSCs (87.55±3.20% of total cells). In contrast, untreated cells showed greater levels of differentiated neurons (βIII-tubulin expressed by 57.12±18.42% of cells) and lower levels of undifferentiated NSCs (nestin was expressed by 58.01±6.20 of total cells). These effects were independent of apoptosis or proliferation.

### Example 9

### Anti-LPA Antibodies in a Murine Cortical Impact Model of Traumatic Brain Injury (TBI)—Treatment

The mouse is an ideal model organism for TBI studies because there is an accepted model of human TBI, the type I IFN system in the mouse is similar to that in human, and the ability to generate gene-targeted mice helps to clarify cause and effect rather than mere correlations. Adult mice were anaesthetised with a single ip injection of Ketamine/Xylazine and the scalp above the parietal bones shaved with clippers. Each scalp was disinfected with chlorhexideine solution and an incision made to expose the right parietal bone. A dentist's drill with a fine burr tip was then used to make a 3 mm diameter circular trench of thinned bone centred on the centre of the right parietal bone. Fine forceps were then used to twist and remove the 3 mm plate of parietal bone to expose the parietal cortex underneath. The plate of bone removed was placed into sterile saline and retained. The mouse was mounted in a stereotaxic head frame and the tip of the impactor (2 mm diameter) positioned in the centre of the burr hole at right angles to the surface of the cortex and lowered until it just touched the dura mater membrane covering the cortex. A single impact injury (1.5 mm depth) was applied using the impactor's computer controller. After impact, the mouse was removed from the head frame and the plate of bone replaced. Bone wax was applied around the edges of the plate to seal and hold the plate in position. The skin incision was then closed with fine silk sutures and the area sprayed with chlorhexideine solution. The mouse was then returned to a holding box underneath a heat lamp and allowed to regain consciousness (total time anaesthetised=30-40 minutes).

Treatments:

Treatments or isotype controls were injected at various time points. Anti-LPA antibody (B3 or other) was injected by tail-IV (0.5 mg). Following 24-48 hours, the animals were humanely sacrificed and their brains analysed.

Analysis:

Neuronal death/survival (TUNEL analysis), reactive astrogliosis (revealed by Ki67-positive cells co-labelled with GFAP), and NS/PC responses (proliferation by CD133/Ki67, migration to the injury site by CD133 and differentiation) were analysed. Immune responses were assessed by CD11b immunostaining. Quantification was performed by density measurement using ImageJ (NIH).

Results:

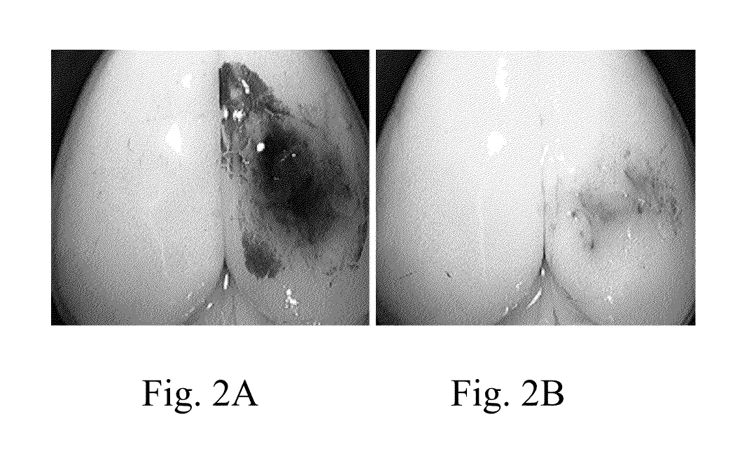

Data from this model showed that anti-LPA antibody (B3) treatment administered before injury reduced the degree of hemorrhage normally seen in the mouse brain following TBI in this cortical impact model. See FIG. 2.

### Example 10

### Anti-LPA Antibodies in Murine Cortical Impact Model of TBI—Prevention

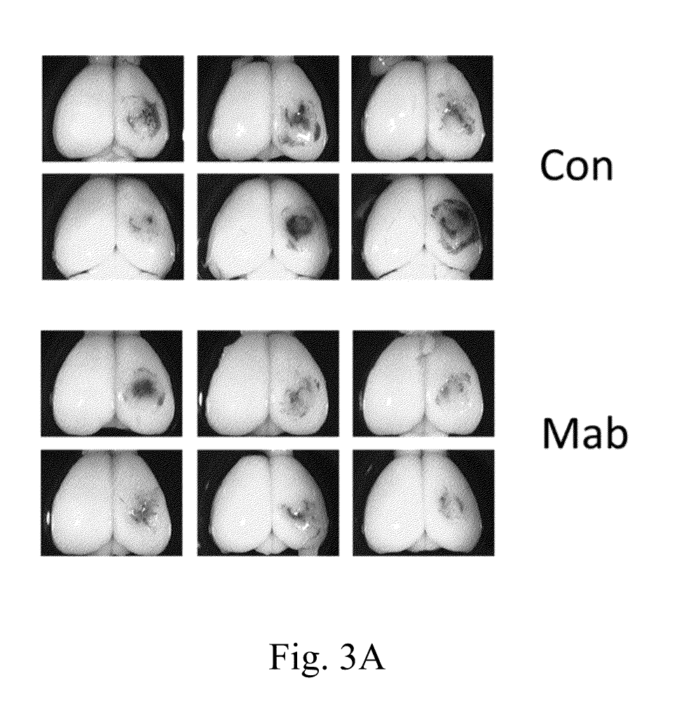

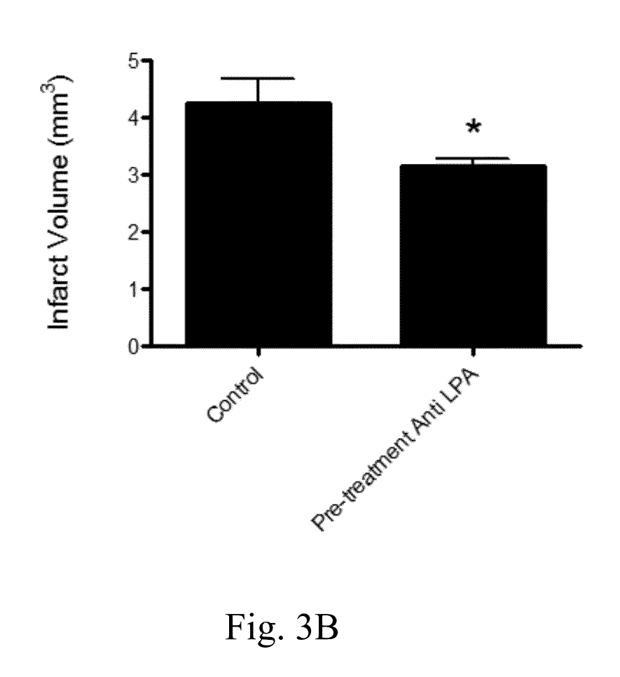

Based on the results of the study described in Example 9, above, a larger double-blinded prevention study using the same murine cortical impact model was undertaken. Here, mice were again subjected to TBI using Controlled Cortical Impact (CCI; described in Example 9, above) and treated with either isotype control monoclonal antibody or anti-LPA antibody B3 given as a single intravenous dose of 0.5 mg antibody (approx. 25 mg/kg) prior to injury. Mice were sacrificed 24 hours later, at which time the infarct size was photographed and its volume quantified. FIG. 3 shows the histological quantitation of infarct size in anti-LPA treated animals vs. isotype control antibody-treated animals. The reduction in brain infarct volume in animals prophylactically treated with anti-LPA antibody compared to control animals was statistically significant.

### Example 11

### Anti-LPA Antibodies in Murine Cortical Impact Model of TBI—Interventional Study #1

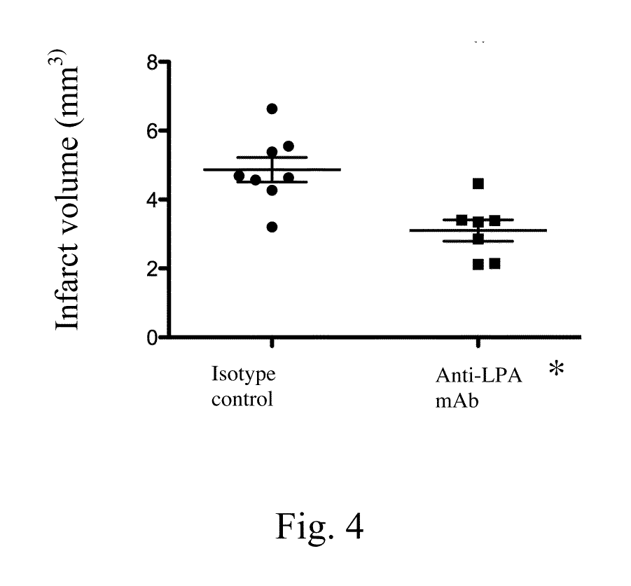

Based on the results of the study described in Example 9, above, a larger double-blinded interventional treatment study was undertaken using the same clinically relevant murine cortical impact model. Mice (8 animals per group) were subjected to TBI using Controlled Cortical Impact (CCI) and treated with either isotype control monoclonal antibody or anti-LPA antibody B3 given as a single intravenous dose of 0.5 mg antibody (approx. 25 mg/kg) 30 minutes after surgery. Mice were sacrificed 48 hours later, at which time the infarct size was photographed and quantified histologically using image analysis. FIG. 4 shows the histological quantitation of infarct size in each anti-LPA treated animals and each isotype control antibody-treated animal. These data show that treatment with an anti-LPA antibody is neuroprotective for TBI, even when given interventionally (after injury).

### Example 12

### Anti-LPA Antibodies in Murine Cortical Impact Model of TBI—Interventional Study #2

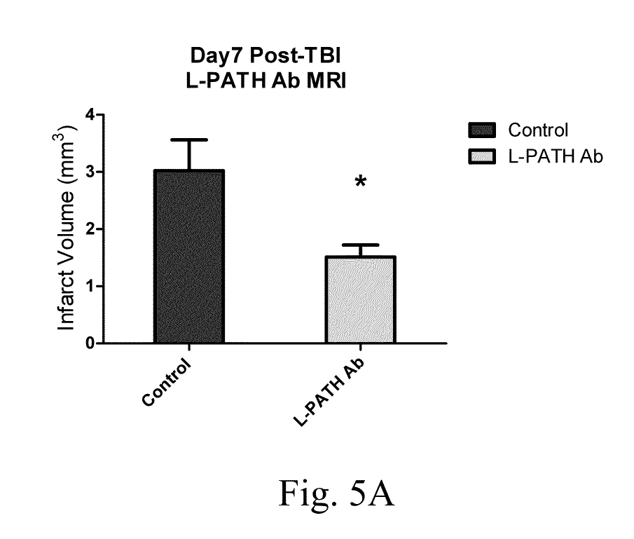

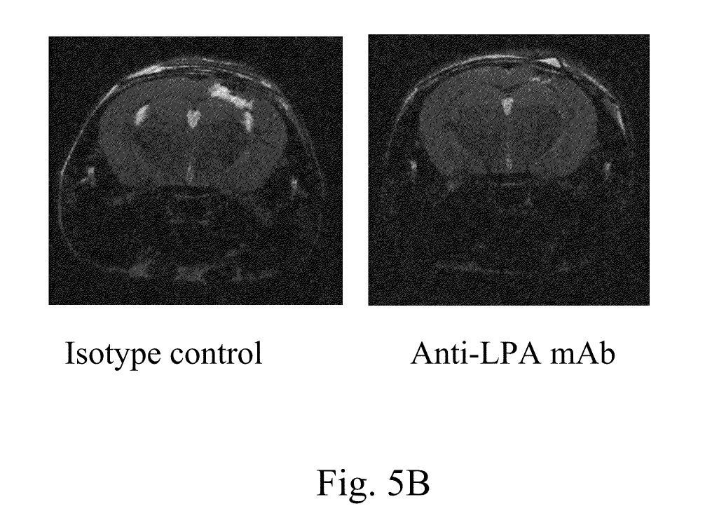

In this double-blinded study, mice (8 per group) were subjected to TBI and treated with an anti-LPA antibody as described above, but here the mice were sacrificed 7 days after injury. Infarct sizes were measured by MRI in this study, and the results are shown in FIG. 5. These results demonstrate a statistically significant decrease in brain infarct size post-TBI in mice treated with an anti-LPA antibody. These data show that treatment with an anti-LPA antibody is neuroprotective for TBI, even when given interventionally after injury. As will be understood, this interventional treatment model is a clinically relevant model.

### Example 13

### Ability of Anti-LPA Antibodies to Improve TBI Functional Outcomes

The previous examples show that anti-LPA antibodies provide significant neuroprotection when given immediately following or prior to brain injury. Anti-LPA antibody treatment has also been shown above to have potential neuroplastic effects (e.g., reduction of glial scarring, enhancement of axonal sprouting, redirection of NS/PCs toward a neuronal cell fate) that may underlie the therapeutic effects. In this example, functional outcomes after TBI were evaluated. The mouse is an excellent species for TBI studies because it is an accepted animal model of human TBI. Marklund, et al. (2006), Curr Pharm Des 12:1645-80.

Experiments were performed in a double-blinded manner, using appropriate isotype-matched mAb controls. Mice were assigned randomly to groups prior to injury. Under isofluorane anesthesia and under aseptic conditions, a moderate level of CCI injury was used to produce a contusion through a 5 mm craniotomy at coordinates: −2 mm Bregma, 3 mm lateral (just behind and lateral to Bregma, over the hindlimb cortex, with some damage to the forelimb cortex), in order to produce a pericontused region over the hind-limb region of the sensory-motor cortex. The injury device consisted of a metal impactor with a 3 mm diameter flat tip that, following stereotactic alignment on the brain, was accelerated onto the intact dural surface using compressed nitrogen at 15 psi to produce a 0.5 mm deformation below the dura. This model consistently produced an injury limited to the cortex down to the level of the corpus callosum. Following injury, the site was sealed with an inert silicone, and the wound was sutured closed. A single dose of antibody (murine anti-LPA monoclonal antibody B3 or isotype control antibody, 25 mg/kg) was administered by penile vein injection at 2 hours post-injury under brief isoflurane anesthesia.

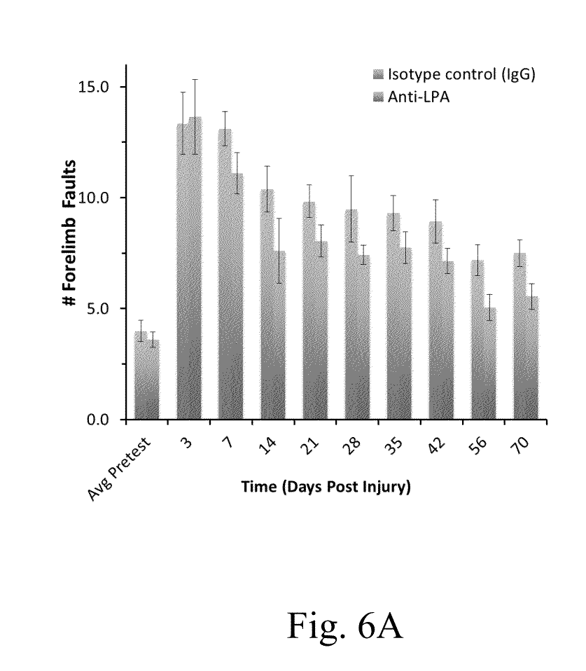

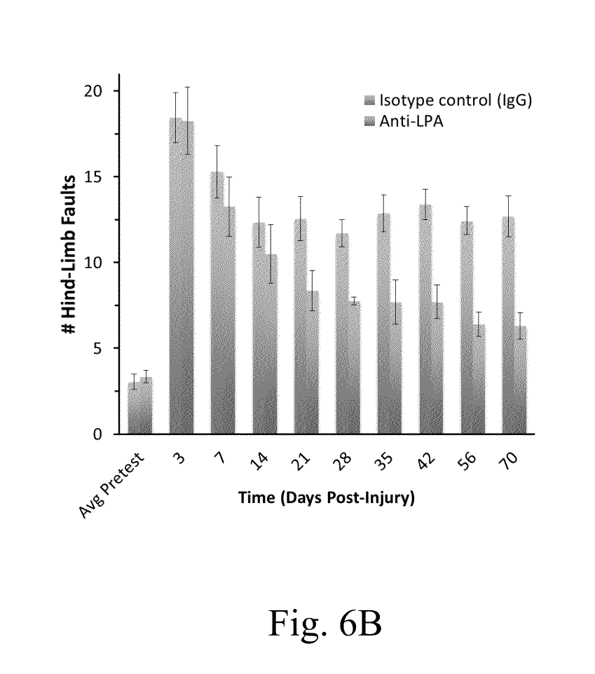

Mice were tested for sensorimotor behavioral readouts before injury and at weekly intervals for 9 or 10 weeks after injury using the grid-walking task and cylinder task. For the grid-walking task, a video camera was used to record the foot-faults of mice exploring a raised, 30 cm by 38 cm wire grid for a 5 minute time period, before and then at weekly intervals after injury. Mice were allowed to walk freely around the grid for three minutes during which a minimum time of two minutes of walking was required. A misstep was counted when the left hind limb paw protruded entirely through the grid with all toes and heel extending below the grid's wire surface. The total number of steps taken with the left hindlimb was also counted. Grid walking was analyzed offline and the number of affected side fore- and hindlimb faults were recorded separately and normalized by the number of total steps taken. The results of this study are shown in FIG. 6. It can be seen that for both forelimb and hindlimb faults (FIGS. 6A and 6B, respectively), the number of faults committed by anti-LPA antibody-treated animals (red bars) was less than the number of faults committed by isotype control antibody-treated animals (blue bars) at all time points after 3 days post-injury.

For the cylinder task, a video camera is used to record the spontaneously rearing behavior of a mouse placed in a 500 ml glass beaker for 5 minutes (or a total of 10 rears) before injury and at weekly intervals after injury. Video is analyzed offline to score the side of initial paw placement. A ratio of left versus right is computed.

MRI images were acquired at one or more time point intervals during the behavioral analysis period. Following sacrifice at 9 or 10 weeks post injury, brains were processed for histology (Nissl stain) and analyzed for volume of brain tissue lost using Stereoinvestigator software (MicroBrightfield, USA) interfaced to a microscope. Volume of tissue lost was calculated by: total ipsilateral volume−total contralateral volume]/total contralateral volume×100. Adjacent sections were immunostained for GFAP visualized with DAB precipitate, and the volume of gliosis was estimated across all brain sections by summing the stained areas and multiplying by the distance between sections.

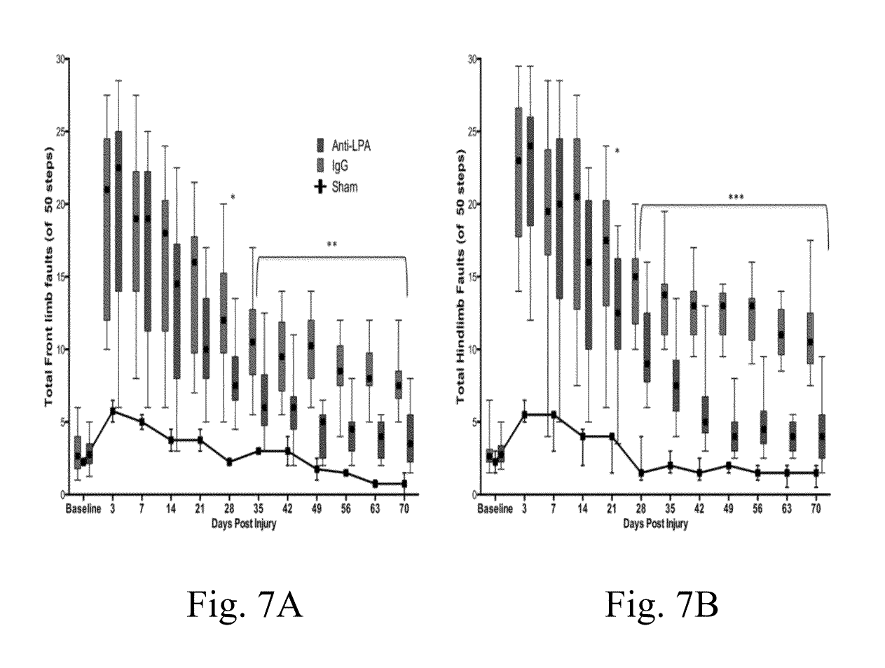

Another grid-walking study was performed in which mice were treated with antibody two hours after CCI, in which post-injury treatment with anti-LPA antibody B3 was found to protect mice from long term functional/behavioral consequences. Larger groups of animals were used to allow robust statistical analysis. Anti-LPA treatment was found to improve long-term sensorimotor function after TBI, as shown in FIG. 7. Total faults were measured of 50 steps for front limbs (FIG. 7A) and hind limbs (FIG. 7B). Data are displayed as median, and 95% confidence intervals. N=5 for anesthetic sham group (black), n=20 for IgG treatment group (blue) and n=25 for anti-LPA antibody treatment group. P-values indicate significant difference between anti-LPA and IgG treatment groups and were obtained by bootstrapping means around the confidence intervals in R; P-values *P<0.05, **P<0.001, ***P<0.0001.

### Example 14

### Neuroprotective Effects of Anti-LPA Antibodies Following Spinal Cord Injury (SCI)

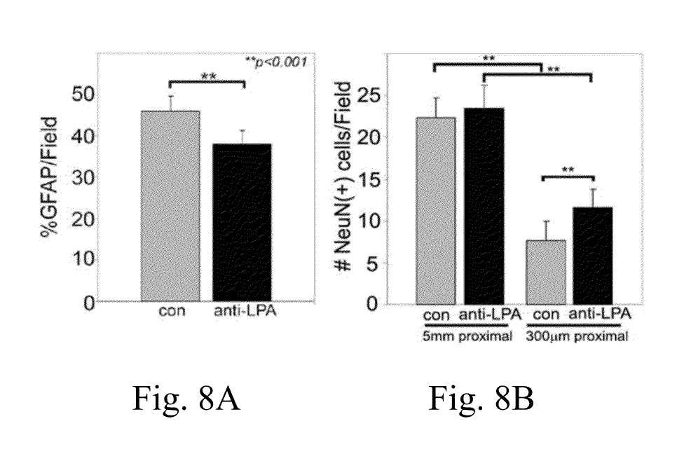

Following SCI as described above (see Example 6), treatment with anti-LPA antibody B3 (0.5 mg/mouse, subcutaneous, twice weekly) for one or two weeks significantly reduced astrocytic gliosis and glial scar formation, as well as neuronal apoptosis. B3 treatment reduced GFAP expression (FIG. 8A) and secretion of chondroitin sulfate proteoglycans (CSPGs), markers for gliosis, into the extracellular matrix by reactive astrocytes at the injury site. Furthermore, B3 antibody treatment also increased neuronal survival at the lesion site, as measured by number of cells staining for NeuN, a neuronal specific nuclear protein (FIG. 8B).

### Example 15

### Functional Recovery in Anti-LPA Antibody-Treated Mice Following SCI

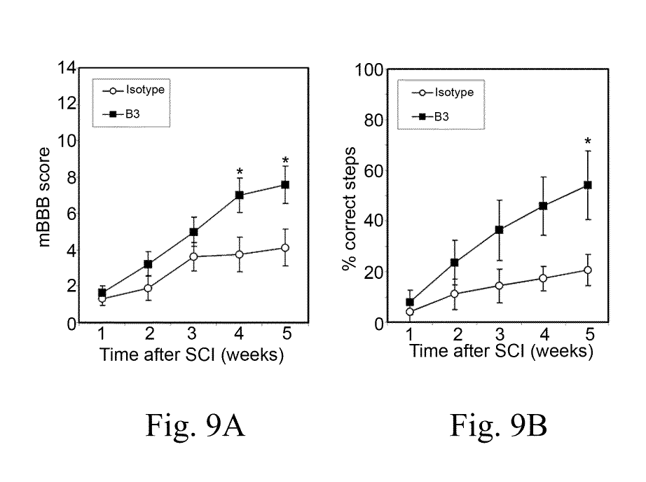

Wildtype mice were given spinal cord hemisection injury as described in Example 6, above. Administration of anti-LPA antibody B3 for two weeks following SCI was found to result in significant functional recovery as determined by open field locomotor test (mBBB) and grid walking test (Goldshmit, et al. (2008), J. Neurotrauma 25(5): 449-465). mBBB is an assessment of hindlimb functional deficits, using a scale ranging from 0, indicating complete paralysis, to 14, indicating normal movement of the hindlimbs. Results are presented as mean+/−SEM. FIG. 9A shows a statistically significant improvement in functional recovery measured by the mBBB at weeks 4 and 5 post-SCI. Mice were also given a grid-walking test (see, e.g., Example 13, above) to assess locomotor function recovery, which combines motor sensory and proprioceptive ability. The test requires accurate limb placement and precise motor control. Intact (uninjured) animals typically cross the grid without making missteps. In contrast, hemisectioned animals make errors with the hindlimb ipsilateral to the lesion. Mice were tested on a horizontal wire grid (1.2×1.2 cm grid spaces, 35×45 cm total area) at weekly intervals following the spinal cord hemisection. Mice were allowed to walk freely around the grid for three minutes during which a minimum time of two minutes of walking was required. Missteps were again counted when the left hind limb paw protruded entirely through the grid with all toes and heel extending below the wire surface. The total number of steps taken with the left hindlimb was also counted. The percentage of correct steps was calculated and expressed +/−SEM. As shown in FIG. 9B, mice treated with anti-LPA antibody B3 showed a dramatic improvement in percent of correct steps in the grid-walking test; this improvement was statistically significant at five weeks post-SCI.

### Example 16

### Antibody to LPA Improves Axonal Regeneration and Neuronal Survival Following SCI

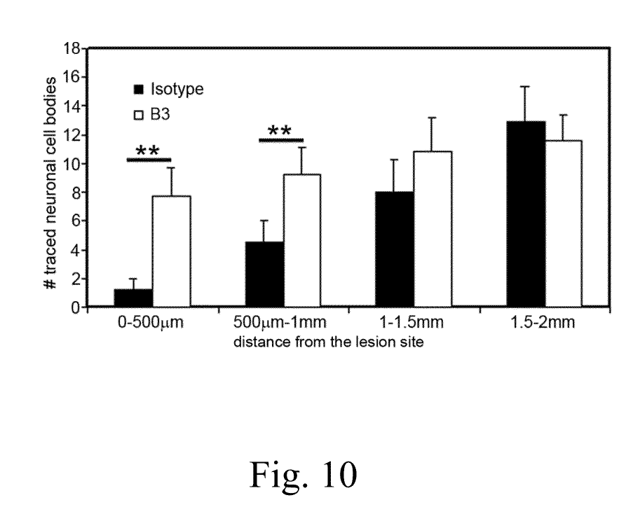

In addition to the functional improvement described in the preceding examples following administration of B3 mAb to wildtype mice for 2 weeks following SCI, anti-LPA antibody treatment also resulted in axonal regeneration through the lesion site and a significant increase in traced neuronal cells that project their processes towards the brain. Tetramethylrhodamine dextran (TMRD) was used to label descending axons that reached the lesion site in isotype controls (n=6) compared to axons that managed to regenerate through the lesion site in B3-treated mice (n=7). Hematoxylin staining was used to reveal the lesion site. Labeled axons also belong to neuronal cells that accumulate label in their cells bodies upstream from the lesion site. Quantitation of number of labeled neuronal cells rostral to lesion site was significantly higher in B3-treated mice (FIG. 10). Data are mean±SEM;**p<0.001. Such neurons may provide later, as part of the plasticity process, a replacement for the loss of long descending or ascending axons after the injury.

### Example 17

### LPA Levels Increase Significantly in Patients after TBI and Correlate with Severity of Injury

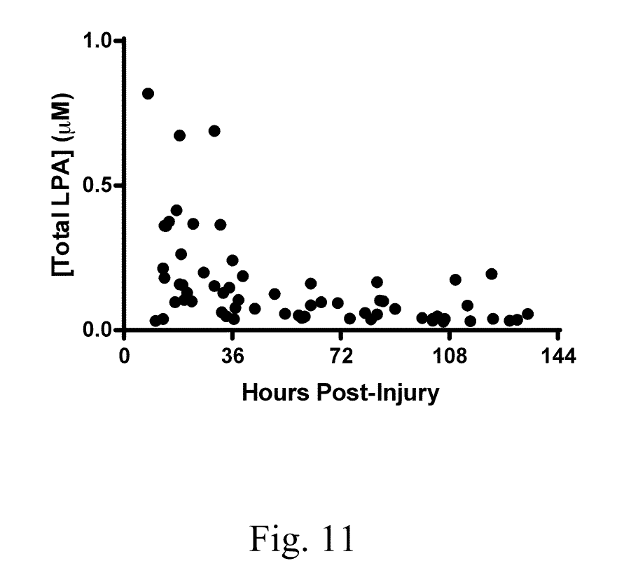

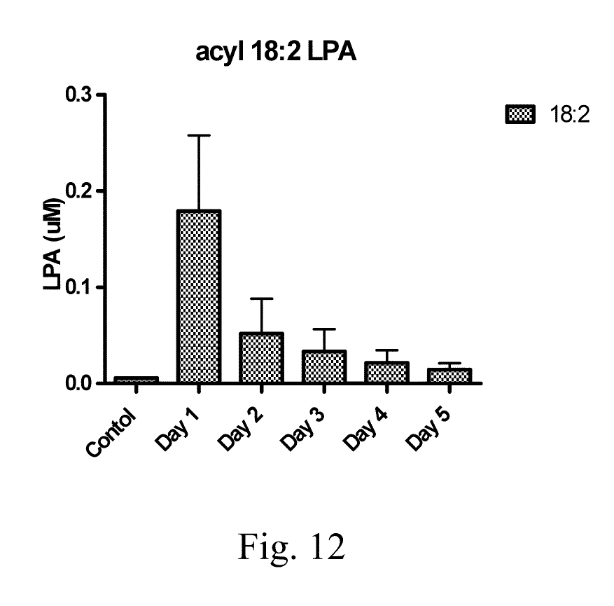

LPA levels have been shown to increase significantly in cerebrospinal fluid (CSF) of human patients after severe traumatic brain injury. Crack, et al. (2014), supra. Those patients had injuries necessitating insertion of an extraventricular drain to monitor intracranial pressure and drain CSF when intraventricular pressure was above a threshold level, thus allowing measurement of CSF in patients within the first few days following a severe brain injury. CSF was drained daily from day of admission (day 0) to day 5, and levels of LPA were measured in the collected CSF by liquid chromatography-mass spectrometry (LC-MS). Patients were also categorized by severity of injury using the Glasgow Coma Scale, Injury Severity Score, presence or absence of hypoxia, and nature of brain injury (i.e., focal or diffuse). Total LPA levels in the CSF of brain-injured patients were found to be elevated (n=26) compared to those in the CSF of patients who had not sustained a brain injury (n=3). A significant and substantial elevation (from 0.05 μM in uninjured control samples to 0.270 μM in brain injured samples) occurred within the first 24 hr after injury and levels returned to basal levels by 120 hr after injury (Crack, et al., 2014). This increase is referred to as the “LPA pulse”. Crack, et al. reported that the 16:0 and 18:0 isoforms of LPA were predominant components of the pulse, though the 18:1, 18:2, and 20:4 isoforms were also elevated during the same period. A scatter plot of total LPA levels over time is shown in FIG. 11 (measurements were made by liquid chromatography-mass spectrometry (LC-MS)); see also FIG. 1 of Crack, et al. (2014). The 18:2 isoform of LPA was also found to increase (pulse) approximately 24 hr post-injury and return to baseline by day 5 (FIG. 12).

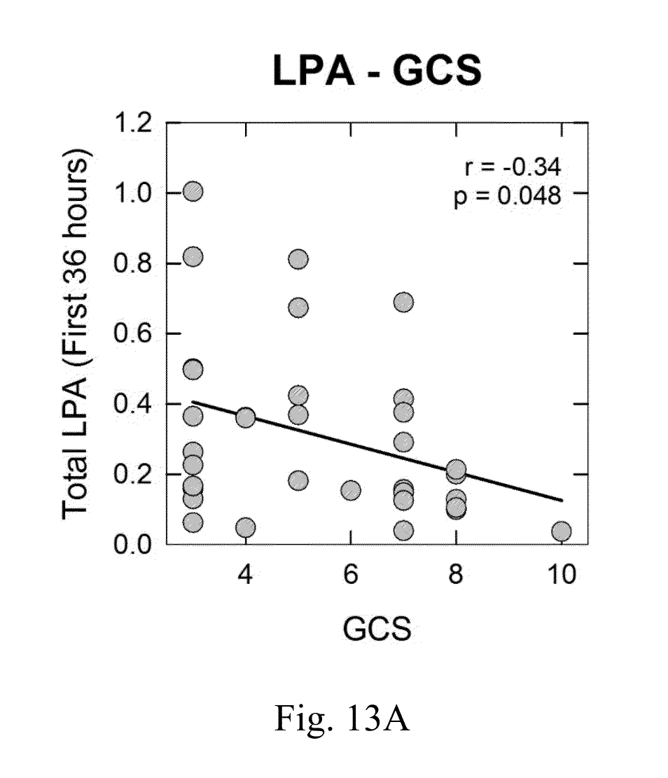

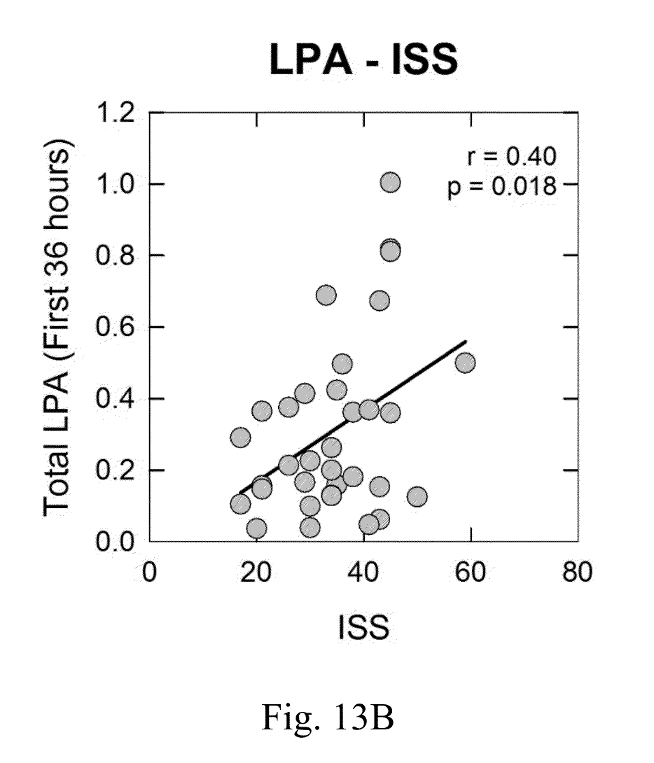

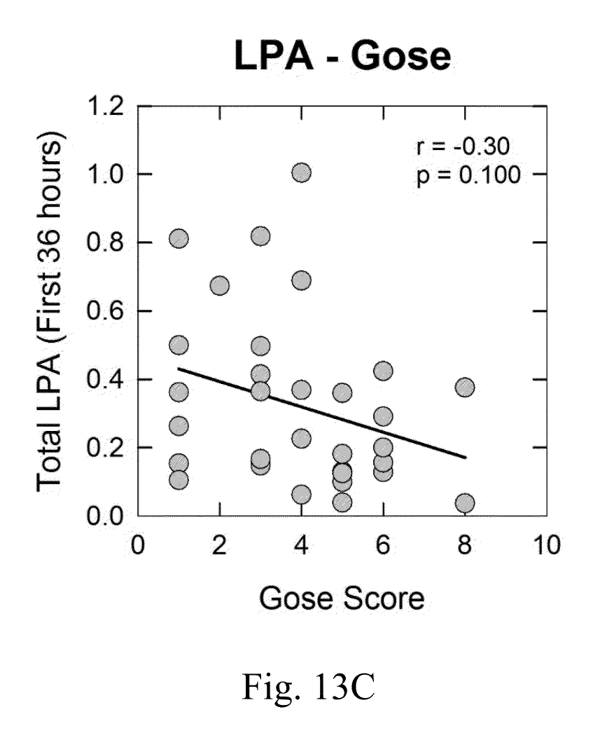

Total LPA (μM) in the CSF from 33 neurotrauma patients collected in the first 36 hr post-injury was plotted against three accepted clinical scores of injury severity, the Glasgow Coma Scale (GCS, FIG. 13A) and Injury Severity Scale (ISS, FIG. 13B) assessed in acute phase, as well as the Extended Glasgow Outcome Scale (GOSE, FIG. 13C) assessed 6 months after injury. These results show that there is a correlation between LPA level and severity of injury in humans, as assessed by all three measures.

These results indicate that in humans, as in mice, a significant increase (pulse) in LPA levels in the CSF is seen at a defined time period following TBI. As the CCI animal model of TBI closely reproduces the closed TBI in the patients described above, the studies described herein strongly predict a positive therapeutic effect of anti-LPA antibodies in treating neurotrauma in humans. Moreover, successful targeting of dysregulated LPA in animals is predictive of success in human conditions in which LPA is also dysregulated. Indeed, the assignee of this application has initiated a Phase 1 human clinical trial of humanized anti-LPA antibody LT3114 in healthy volunteers. It is expected that that Phase 1 study will be followed by a Phase 1b/2a trial in patients with severe TBI.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for treating neurotrauma, comprising administering to a subject having or suspected of having sustained neurotrauma a therapeutically effective amount of an antibody, or fragment thereof, that binds lysophosphatidic acid (LPA), thereby treating said neurotrauma, wherein the antibody or LPA-binding fragment thereof comprises:
a. at least one heavy chain variable domain comprising an amino acid sequence: EVQLVQSGAEVKKPGESLKISCQAFGDAFTNYLIEWVRQMPGQGLEWIGLIYPDSGYINYNENFKGQATLSADRSSS TAYLQWSSLKASDTAMYFCARRFAYYGSGYYFDYWGQGTMVTVSS (SEQ ID NO: 61); and
b. at least one light chain variable domain comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).

2. A method according to claim 1 wherein the neurotrauma is selected from the group consisting of traumatic brain injury, stroke, brain or spinal cord hemorrhage, brain infarct, and spinal cord injury.

3. A method according to claim 1 wherein the subject is a human subject.

4. A method according to claim 1 wherein the antibody or fragment thereof comprises:
two heavy chain variable domains each comprising an amino acid sequence: EVQLVQSGAEVKKPGESLKISCQAFG DAFTNYLIEWVRQM PGQGLEWIGLIYPDSGYI NYN EN FKGQATLSADRSSS TAYLQWSSLKASDTAMYFCARRFAYYGSGYYFDYWGQGTMVTVSS (SEQ ID NO: 61); and
two light chain variable domains each comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).

5. A method for reducing the size of a brain infarct in a subject having or suspected of having sustained a traumatic brain injury, comprising administering to said subject a therapeutically effective amount of an antibody, or fragment thereof, that binds lysophosphatidic acid, thereby reducing the size of said brain infarct, wherein the humanized antibody or fragment thereof comprises:
a. at least one heavy chain variable domain comprising an amino acid sequence: EVQLVQSGAEVKKPGESLKISCQAFGDAFTNYLIEWVRQMPGQGLEWIGLIYPDSGYINYNENFKGQATLSADRSSS TAYLQWSSLKASDTAMYFCARRFAYYGSGYYFDYWGQGTMVTVSS (SEQ ID NO: 61); and
b. at least one light chain variable domain comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).

6. A method according to claim 5 wherein the subject is a human subject.

7. A method according to claim 5 wherein the humanized antibody or fragment thereof comprises:
two heavy chain variable domains each comprising an amino acid sequence: EVQLVQSGAEVKKPGESLKISCQAFGDAFTNYLIEWVRQMPGQGLEWIGLIYPDSGYINYNENFKGQATLSADRSSS TAYLQWSSLKASDTAMYFCARRFAYYGSGYYFDYWGQGTMVTVSS (SEQ ID NO: 61); and
two light chain variable domains each comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).

8. A method for of increasing locomotor function recovery in a subject having sustained a neurotrauma resulting in a decrease in locomotor function, comprising administering to said subject a therapeutically effective amount of an antibody, or fragment thereof, that binds lysophosphatidic acid, thereby increasing the locomotor function recovery of the subject, wherein the humanized antibody or fragment thereof comprises:
a. at least one heavy chain variable domain comprising an amino acid sequence: EVQLVQSGAEVKKPGESLKISCQAFGDAFTNYLIEWVRQMPGQGLEWIGLIYPDSGYINYNENFKGQATLSADRSSS TAYLQWSSLKASDTAMYFCARRFAYYGSGYYFDYWGQGTMVTVSS (SEQ ID NO: 61); and
b. at least one light chain variable domain comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).

9. A method according to claim 8 wherein the subject is a human subject.

10. A method according to claim 8 wherein the humanized antibody or fragment thereof comprises:
two heavy chain variable domains each comprising an amino acid sequence: EVQLVQSGAEVKKPGESLKISCQAFGDAFTNYLIEWVRQMPGQGLEWIGLIYPDSGYINYNENFKGQATLSADRSSS TAYLQWSSLKASDTAMYFCARRFAYYGSGYYFDYWGQGTMVTVSS (SEQ ID NO: 61); and
two light chain variable domains each comprising an amino acid sequence: DVVMTQTPLSLPVTPGEPASISCRSSQSLLKTNGNTYLHWYLQKPGQSPKLLIFKVSNRFSGVPDRFSGSGSGTDFT LKISRVEAEDVGVYFCSQSTHFPFTFGQGTKLEIK (SEQ ID NO: 42).
